(19) United States
(12) Patent Application Publication
(10) Pub. No.: US 2017/0068795 A1
(43) Pub. Date: Mar. 9, 2017

(54) Compositions and Methods for Assessing Risk of Autism Spectrum Disorder During Early Childhood

(71) Applicant: The Penn State Research Foundation, University Park, PA (US)

(72) Inventors: Guodong Liu, Hummelstown, PA (US); Lan Kong, Hummelstown, PA (US); Douglas L. Leslie, Hummelstown, PA (US); Michael Murray, Middletown, PA (US)

(21) Appl. No.: 15/259,920

(22) Filed: Sep. 8, 2016

Related U.S. Application Data
(60) Provisional application No. 62/215,407, filed on Sep. 8, 2015.

(51) Int. Cl. G06F 19/00; A61B 5/16; A61B 5/00

(52) U.S. Cl. CPC G06F 19/3431; G06F 19/322; A61B 5/4076; A61B 5/7275; A61B 5/16

(57) ABSTRACT

The invention includes an early detection system for ASD that combines the assessment of various factors and comorbid conditions to accurately generate a prediction model for assessing the degree of ASD.

### CROSS-REFERENCE TO RELATED APPLICATIONS

This application claims priority to U.S. Provisional Patent Application No. 62/215,407 filed Sep. 8, 2015, the contents of which are incorporated by reference herein in their entirety.

### BACKGROUND OF THE INVENTION

Autism Spectrum Disorder (ASD) is a spectrum of complex brain disorders manifested by difficulties in verbal/non-verbal social interactions and patterns of repetitive behaviors. The prevalence of ASD has been on the rise at an alarming rate. According to a report of the Centers for Disease Control and Prevention (CDC) in March 2014, one out of every 68 children in the U.S. has ASD. Despite the fact that there has been mounting evidence suggesting that ASD can be traced back to as early as the second trimester of pregnancy, ASD diagnosis has been consistently late, with most diagnoses at the age of 4 years or older. While early intervention is critical, its optimal outcome cannot be achieved without early detection.

Typically only specialists, such as a pediatric psychiatrist can make reliable ASD diagnosis, using a battery of behavior-based tests. Constrained by medical cost as well as insurance reimbursement, these tests are not for every kid, but reserved only for kids who have already shown certain ASD behavioral tendencies. However, most of these behavioral signs won't be reliably identified or assessed by parents or general pediatricians until a child reaches 3 years old, if not later, so they can be referred to a specialist for diagnosis. By then, it is already too late for effective early intervention. In order to advance early detection, it is imperative to identify some other types of early markers of ASD predicting value.

Thus, there is an urgent need in the art for compositions and methods for early detection of ASD. The present invention addresses these needs.

### SUMMARY OF THE INVENTION

The present invention provides compositions and methods for assessing the degree of severity of Autism Spectrum Disorder (ASD) in a subject. In one embodiment, the method comprises determining the incidence of one or more medical conditions in a subject, comparing the incidence of the one or more medical conditions with a comparator control, and assessing the subject's degree of severity of ASD based on the subject's level of incidence the one or more medical conditions compared to the comparator control.

In one embodiment, the method further comprises the step of diagnosing the subject with ASD based on the subject's level of incidence of the one or more medical conditions compared to the comparator control.

In one embodiment, the one or more medical condition is selected from the group consisting of unspecified hearing loss, Otitis Media, epilepsy, lack of coordination, influenza with other manifestation, patent ductus arteriosus, dysphagia, abdominal pain, convulsion, ear pain, teething syndrome, delayed milestone, weight/metabolism issue, disorder due to short gestation/low weight, lingering issue from labor complications, respiratory abnormalities, asthma, delay in development, preterm infants, rash and other non-specified skin eruption, constipation, diarrhea, contact dermatitis and other eczema, atopic dermatitis and related conditions, lack of normal physiological development, vomiting, acute laryngitis and trachetitis, feeding difficulty, colitis, viral infection, acute pharyngitis, cough, GERD, fever, acute upper respiratory infection, and any combination thereof.

In one embodiment, determining the incidence of one or more medical conditions includes identifying a health pattern for the subject.

In one embodiment, the subject is less than 6 months old, and the health pattern includes incidence of epilepsy and otitis media.

In one embodiment, the health pattern is dynamic and varies over time depending on the subject's age.

In one embodiment, the health pattern is individualized by being assessed for each individual subject.

In one embodiment, the health pattern includes a pattern of incidence of at least two of: respiratory system disease, abnormal weight or physical development, digestive system disorder, disorder relating to short gestation or low body weight, or neonatal labor complication.

In one embodiment, the subject is less than six months of age.

In one embodiment, the subject is less than ninety days of age.

The invention also provides implementing the assessment of the severity of ASD in an electronic format such as a computer. Accordingly, the step of assessing the severity of ASD can be carried out using a computer.

In one embodiment, the one or more steps are carried out in connection with an Electronic Medical Records (EMR) database, and further including a step of generating a notice if the step of assessing indicates that one or more subject whose records are in the EMR may have a severity of ASD that is above a specified degree.

In one embodiment, the one or more steps of the invention are carried out on a software application or website platform.

In one embodiment, the one or more of the steps of the invention are carried out using a subscription software application.

In one embodiment, the method of assessment is carried out once per transaction.

In one embodiment, the information regarding the incidence of one or more medical conditions is individualized by inputting information regarding one individual subject.

In one embodiment, the methods of the invention further comprises the step of providing the subject with a medical treatment based upon the step of assessing.

In one embodiment, the subject is up to 36 months of age and the medical treatment is early intervention therapy to improve the subject's ability to communicate, locomote, or socialize.

In one embodiment, the medical treatment is selected from the group consisting of behavioral, communications, dietary, medication, complementary medicine, alternative medicine, and any combination thereof.

In one embodiment, the incidence of one or more medical conditions is used to identify a health pattern for the subject, and the medical treatment is individualized for a specific subject based upon the health pattern.

In one embodiment, the compositions and methods of the invention for assessing the severity of ASD can be incorporated into educational platforms. In one embodiment, the method of the invention further comprises the step of providing educational information regarding ASD based upon the step of assessing.

In one embodiment, the incidence of one or more medical conditions is used to identify a health pattern for the subject, and the educational information regarding ASD is individualized for a specific subject based upon the health pattern.

In one embodiment, the method of the invention can be incorporated into a platform that can recalibrate the incidence of one or more medical conditions and/or comparator control to provide another level of assessment. For example, the method of the invention can be incorporated into a system that can perform an on-going assessment including but is not limited to assessment refinement, machine learning and the like.

In one embodiment, the one or more steps are dynamic, comprising periodically: evaluating results of the assessing, and then refining one of more of the step of determining, comparing and assessing based on the evaluating.

In one embodiment, the comparator control is periodically updated.

The invention also provides a method of diagnosing ASD in a subject. In one embodiment, the method comprises determining the incidence of at least one medical condition in a subject, comparing the incidence of the at least one medical condition with a comparator control, and diagnosing the subject with ASD when the level of the at least one medical condition is present at a statistically significant amount when compared with the comparator control.

The invention also provides a method for assessing the risk of developing Autism spectrum disorder (ASD) in a subject. In one embodiment, the method comprises determining the incidence of at least one medical condition in a subject, comparing the incidence of the at least one medical condition with a comparator control, and assessing the subject's risk of developing ASD when the level of the at least one medical condition is present at a statistically significant amount when compared with the comparator control.

The invention also provides a system for diagnosing ASD in a subject. In one embodiment, the system comprises a component to assess the presence of at least one medical condition in a subject.

The invention also provides a method for diagnosing risk of developing ASD in a subject, the method comprising determining the number of emergency department (ED) visitations by a subject during the first six months of life, and diagnosing the subject as having an increased risk of developing ASD when the number of emergency department visitations by the subject is greater than two. In one embodiment, the method further comprises providing the subject with an ASD evaluation or ASD therapy based upon increased ED utilization. In various embodiments, the therapy may be one of early intervention therapy, pivotal response treatment (PRT), verbal behavior (VB) therapy, applied behavioral analysis (ABA), discrete trial teaching (DTT), floortime (DIR), relationship development intervention (RDI), training and education of autistic and related communication handicapped children (TEACCH), social communication/emotional regulation/transactional support (SCERTS), speech-language therapy (SLT), occupational therapy (OT), sensory integration (SI), physical therapy (PT), social skills therapy, picture exchange communication system (PECS), auditory integration therapy (AIT), gluten free casein free diet (GFCF), early start Denver model (ESDM), behavioral therapy, communication therapy, dietary therapy, drug therapy, complementary medicine therapy, alternative medicine therapy or any combination thereof.

The invention also provides a method for diagnosing risk of developing ASD in a subject, the method comprising determining the number of ED visitations by a subject, comparing the number of ED visitations with a comparator control, and diagnosing the subject as having an increased risk of ASD when the number of ED visitations by the subject is statistically significantly greater when compared with the comparator control. In one embodiment, the method further comprises providing the subject with a medical therapy based upon ED utilization. In various embodiments, the therapy may be one of occupational therapy, behavioral therapy, communication therapy, dietary therapy, drug therapy, complementary medicine therapy, alternative medicine therapy, and any combination thereof.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts a breakdown of the childhood ASD and non-ASD sub-cohorts by gender, and geographic region.

FIG. 2 is a graph showing comorbid medical condition incidence ratios within 180 days after birth.

FIG. 3 depicts a breakdown of the adolescent ASD and non-ASD sub-cohorts by year including the average age and gender for each year.

FIG. 4 depicts a breakdown of the adolescent ASD and non-ASD sub-cohorts by age range, geographic region, healthcare plan and locality (urban vs rural).

FIG. 5, comprising FIG. 5A through FIG. 5C, depicts charts showing the annual percentage of patients requiring Emergency Department (ED) utilization. FIG. 5A depicts a chart showing an increase in annual percentage of ASD patients who had ED visit(s). FIG. 5B depicts the percentage of patients with ED visits among ASD vs. non-ASD adolescent populations. FIG. 5C depicts an increase in number of ASD adolescents with ED visits from 2005 to 2013 (baseline number at 2005=224 cases).

FIG. 6, comprising FIG. 6A through FIG. 6F, depicts analyses of the proportion of ED patients between ASD and non-ASD sub-populations. FIG. 6A and FIG. 6B depict the proportion of non-ASD and ASD ED patients respectively, broken down by age group. FIG. 6C and FIG. 6D depict the proportion of non-ASD and ASD ED patients respectively, broken down by gender. FIG. 6E and FIG. 6F depict the proportion of non-ASD and ASD ED patients respectively, broken down by type of residence, i.e. rural vs. urban.

FIG. 7 depicts an analysis of the proportion of ED patients who received behavioral health services during ED visits annually.

FIG. 8 depicts results showing a significant difference in ED utilization by ASD vs non-ASD, by older adolescents vs younger adolescents, and by females vs males.

FIG. 9 depicts a breakdown of the ASD and non-ASD cohorts used to generate a profile on healthcare utilization during the first year of life in children with ASD.

FIG. 10 depicts results showing the healthcare utilization during the 1^{st} year of life in children with and without ASD.

FIG. 11 depicts the adjusted odds ratios of ASD risk for various covariates.

### DETAILED DESCRIPTION

The present invention provides a system for early detection of Autism Spectrum Disorders (ASD). In one embodiment, the system is a comprehensive and objective ASD risk assessment on children from birth to about 5 years old based on models developed and trained using large databases of electronic medical records (EMRs), as well as meta-analysis of other previous relevant studies.

In one embodiment, the invention provides a system for assessing a degree of severity of ASD. In one embodiment, assessing the degree of ASD comprises determining the incidence of one or more medical conditions in a subject, comparing the incidence of the one or more medical conditions with a comparator control, and assessing the subject's degree of severity of ASD based on the subject's level of incidence the one or more medical conditions compared to the comparator control.

In one embodiment, the system of the invention can assess ASD severity in children younger than 6 months. This ASD severity assessment allows for proportional treatment of ASD.

In one embodiment, the invention provides an ASD assessment model to provide an evidence-based means of determining ASD severity in children and to identify changes in ASD severity over time.

In one embodiment, the system of the invention can detect high ASD-risk children younger than 6-months. This early detection of ASD allows for early treatment of ASD.

In one embodiment, the invention provides an ASD prediction model to provide an evidence-based means of early-warning system for potential children of high ASD risk and to identify subjects for further psychiatric specialist evaluation for ASD well before classic ASD symptoms can be picked up by an observer (e.g., and ordinary observer, general healthcare practitioner, and the like).

In one embodiment, the system of the invention is based on an observer trained by using national databases of comprehensive medical histories of large populations of children and their families, from pregnancy, through birth, to early childhood, plus other genetic and demographic information, such as race, gender, parental ages at childbirth, family history (e.g. if sibling has ASD). This system can accurately assess a child's risk of ASD in terms of a probability, which primary care physicians (PCP) as well as parents can use as an evidence for requesting for specialist screening.

In one embodiment, the invention provides a system for early detection in a form of a standalone device, such as a hand-help device application, or a web-based evaluation tool.

In one embodiment, the invention provides a personalized ASD risk prediction tool to help observers make an informed, evidence-based decision for early detection of ASD. One advantage is that the system provides parents not trained in the health profession to make an informed, evidence-based decision for early detection of ASD.

In one embodiment, the system of the invention comprises factors or indicators that can discriminate between a normal subject and a subject at risk of developing ASD or having ASD. The factors of the invention can be used to screen, assess risk, assess severity, diagnose and monitor the onset or progression of ASD. The factors or indicators of the invention form part of a subject health profile and can be used to establish and evaluate treatment plans against ASD.

In one embodiment, the invention provides a system that combines various factors and comorbid conditions to generate a kit or prediction model for the diagnosis of ASD. Examples of factors and comorbid conditions include but are not limited to ear infection, respiratory system issues, gastrointestinal issues, epilepsy, feeding difficulty, weight/development/metabolism issues, disorders due to short gestation/low weight, new born affected from labor complications, lack of coordination, convulsion, skin issues such as eczema, rash, skin eruption and dermatitis, and viral infection.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass non-limiting variations of ±40% or ±20% or ±10%, ±5%, ±1%, or ±0.1% from the specified value, as such variations are appropriate.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

“Autism spectrum disorders (ASDs)” as used herein refer to complex neurodevelopmental disabilities characterized by stereotypic behaviors and deficits in communication and social interaction. The term “spectrum” refers to the wide range of symptoms, skills, and levels of impairment, or disability, that patients with ASD can have. ASD is generally diagnosed according to guidelines listed in the Diagnostic and Statistical Manual of Mental Disorders, Fourth Edition—Text Revision (DSM-IV-TR). The manual currently defines five disorders, sometimes called pervasive developmental disorders (PDDs), as ASD, including Autistic disorder (classic autism), Asperger's disorder (Asperger syndrome), Pervasive developmental disorder not otherwise specified (PDD-NOS), Rett's disorder (Rett syndrome), and Childhood disintegrative disorder (CDD). Some patients are mildly impaired by their symptoms, but others are severely disabled. ASD encompasses a set of complex disorders with poorly defined etiologies, and no targeted cure.

As used herein, the term “marker” or “factor” or “indicator” is meant to include a parameter which is useful according to this invention for determining the presence and/or ASD.

The level or frequency of a marker “significantly” differs from the level or frequency of the marker in a subject if the level or frequency of the marker in a subject differs from the level or frequency in a sample from a reference subject, population or comparator control by an amount greater than the standard error of the assay employed to assess the marker, and preferably at least 10%, and more preferably 25%, 50%, 75%, or 100%.

The term “comparator control” relates to a level of a marker or frequency of an event in a subject, or a population, with or without a diagnosis of ASD, such that the control or reference standard may serve as a comparator against which a subject can be compared.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of at least one sign or symptom of the disease or disorder, the frequency with which such a sign or symptom is experienced by a patient, or both, is reduced.

As used herein, the term “diagnosis” refers to the determination of the presence of a disease or disorder, such as ASD. In some embodiments of the present invention, methods for making a diagnosis are provided which permit determination of the presence of a disease or disorder, such as ASD.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

“Measuring” or “measurement,” or alternatively “detecting” or “detection,” means assessing the presence, absence, frequency, quantity or amount (which can be an effective amount) of a given marker, indicator or factor in subject, including the derivation of qualitative or quantitative levels of such, or otherwise evaluating the values or categorization of a subject's clinical parameters.

The terms “normal” and “healthy” are used herein interchangeably. They include an individual or group of individuals who do not have a diagnosis of ASD. The term “normal” is also used herein to qualify a health profile from a healthy individual.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “providing a prognosis” refers to providing a prediction of the probable course and outcome of ASD, including prediction of severity. The methods can also be used to devise a suitable therapeutic plan, e.g., by indicating whether the subject would benefit from pharmaceutical treatments and/or non-pharmaceutical intervention services.

A “reference level” of a comparator control means a level that is indicative of a particular disease state, phenotype, or lack thereof, as well as combinations of disease states, phenotypes, or lack thereof. A “positive” reference level means a level that is indicative of the presence of or increased risk of developing a particular disease state or phenotype. A “negative” reference level means a level that is indicative of a lack of or a low risk of developing a particular disease state or phenotype.

The term “risk stratification,” according to the invention, comprises finding patients, particularly those having ASD, for the purpose of diagnosis and therapy/treatment of ASD, with the goal of allowing as advantageous a course of the ASD therapy/treatment as possible.

“Sample” or “biological sample” as used herein means a biological material isolated from an individual. The biological sample may contain any biological material suitable for detecting desired marker, factor or indicator, and may comprise cellular and/or non-cellular material obtained from the individual.

“Standard control value” as used herein refers to a predetermined frequency of an event within a control population, either in the whole population or a sub-set of the control population that is matched on the basis of one or more characteristics (e.g. age, ethnicity, gender). The standard control value is suitable for the use of a method of the present invention, in order for comparing the level of incidence of a comorbidity of a subject with that of a control population. An established standard control for a person without ASD provides information including a standard developmental profile, a standard behavioral profile, and a standard health profile that is typical for an average, healthy person of reasonably matched background, e.g., gender, age, ethnicity, and medical history.

The terms “treatment” and “therapy” may be used interchangeably. The word “intervention” may also be used to describe a treatment or therapy.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based on the identification of particular medical conditions and/or comorbidities in ASD. Accordingly, the invention includes an early detection system for ASD that combines the assessment of various factors and comorbid conditions to accurately generate a prediction model for the diagnosis of ASD. Examples of factors and comorbid conditions include but are not limited to ear infection, respiratory system issues, gastrointestinal issues, epilepsy, weight/development/metabolism issues, disorders due to short gestation/low weight, and new born affected from labor complications.

The invention is partly based on the identification of particular medical conditions that are highly prevalent in early childhood.

In one embodiment, the invention provides compositions and methods for assessing the degree of severity of ASD. In some instances, assessing the severity of ASD identifies subjects that can be diagnosed with ASD. The diagnosis of ASD can be performed by the same or different person that is assessing the severity of ASD in the subject. In other instances, assessing the severity of ASD identifies subjects that are not considered to have ASD.

In one embodiment, the assessment is made based on multiple factors. As discussed elsewhere herein, these multiple factors can be part of a “health profile.”

On advantage of the invention is that the assessment can be performed by any person and can be performed at a very early stage in a subject's life, and thereby permitting early intervention which may be key to better outcomes.

### Identifying a Factor/Indicator/Marker

The invention includes methods for the identification of differentially prevalent factors between normal and ASD as risk subjects or subjects having ASD, as well as methods for the detection of the differentially prevalent factors.

In one embodiment, identifying early clinical indicators of ASD involves a comprehensive and objective ASD assessment on children from birth to about 5 years old based large databases of electronic medical records (EMRs), as well as meta-analysis of other previous relevant studies. Assessment of data from claims database, census, surveys, and the like is also performed to identify indicators of early detection of ASD or to identify severity of ASD. For example, a sample size of at least 100,000 children with medical records from birth up to 5 years old is assess to identify an indicator of ASD.

Controls groups may either be normal subjects or subjects from known stages of ASD. As described elsewhere herein, comparison of the patterns of the subject to be tested with those of the controls can be used to diagnose between normal and being at risk of ASD or severity of ASD. In some instances, the control groups are only for the purposes of establishing initial cutoffs for the assays of the invention. Therefore, in some instances, the systems and methods of the invention can diagnose between normal subjects and ASD at risk subjects or subjects having ASD without the need to compare with a control group.

The present inventors have performed a medical record comprehensive association study on several large patient cohorts and have successfully identified a number of comorbidities associated with ASD. Thus, in accordance with the present invention, kits are provided for performance of a method for detecting a propensity for developing ASD is provided. An exemplary kit comprises means for assessing the subject for the presence or absence of a comorbidity associated with ASD in a subject, wherein if a particular comorbidity is present, the subject has an increased risk for developing ASD. In one embodiment, the presence of a particular comorbidity is selected from the group of ear infection, respiratory system issues, gastrointestinal issues, epilepsy, feeding difficulty, weight/development/metabolism issues, disorders due to short gestation/low weight, new born affected from labor complications, lack of coordination, convulsion, skin issues such as eczema, rash, skin eruption and dermatitis, and viral infection.

In one embodiment, the presence of a particular comorbidity is selected from the group of unspecified hearing loss, lack of coordination, influenza with other manifestation, patent ductus arteriosus, dysphagia, abdominal pain, convulsion, ear pain, teething syndrome, delayed milestone, respiratory abnormalities, asthma, delay in development, preterm infants, rash and other non-specified skin eruption, constipation, diarrhea, contact dermatitis and other eczema, atopic dermatitis and related conditions, lack of normal physiological development, vomiting, acute laryngitis and trachetitis, feeding difficulty, colitis, viral infection, acute pharyngitis, cough, GERD, fever, Otitis Media, acute upper respiratory infection, and the like. In another embodiment, an established list of comorbid medical conditions may be used to identify additional medical conditions that are comorbid with ASD. For example, if a population of subjects sharing known comorbidities also share an additional medical condition and the incidence of the additional medical condition is statistically significant, then the additional medical condition may be also identified as comorbid with ASD.

In one embodiment, a diagnosis of ASD is likely when epilepsy and otitis media are both identified in a child less than 6 months old. Diagnosis of ASD or severity of ASD may increase if the child is identified to have had at least two incidences of different medical conditions among respiratory system disease, unspecified fever, abnormal weight or physical development, digestive system disorder, disorder relating to short gestation or low body weight, neonatal labor complications, lack of coordination, convulsion, skin issues such as eczema, rash, skin eruption and dermatitis, and viral infection.

In one embodiment, a comorbidity is an increased level of ED utilization. In one embodiment, the increased level of ED utilization is identified in an adolescent. In one embodiment, the increased level of ED utilization is identified in a child less than 6 months old.

### Methods

In one embodiment, the present invention relates to the identification of factors including comorbidities associated with ASD to generate a health profile for a subject. Accordingly, the present invention features methods for identifying subjects who are at risk of developing ASD, including ASD-related conditions, or for assessing the severity of ASD by detection of the factors and assessing the health profile disclosed herein. These factors or otherwise health profile are also useful for monitoring subjects undergoing treatments and therapies for ASD and ASD related conditions, and for selecting or modifying therapies and treatments that would be efficacious in subjects having ASD and ASD related conditions, wherein selection and use of such treatments and therapies slow the progression of ASD and ASD related conditions or prevent their onset.

The invention provides improved diagnosis and prognosis of ASD. The risk of developing ASD or the severity of ASD can be assessed by measuring one or more of the factors described herein, and comparing the presence and values of the factors to reference or index values. Such a comparison can be undertaken with mathematical algorithms or formula in order to combine information from results of multiple individual factors and other parameters into a single measurement or index. Subjects identified as having an increased risk of ASD can optionally be selected to receive treatment regimens, such as administration of prophylactic or therapeutic compounds or implementation of therapy (e.g. behavioral therapy, communication therapy, occupational therapy, and early intervention therapy), exercise regimens or dietary supplements to prevent, treat or delay the onset of ASD. Subjects with ASD can optionally be selected to receive treatment regimens that are proportional to their individual ASD severity.

Identifying a subject before they develop ASD enables the selection and initiation of various therapeutic interventions or treatment regimens in order to delay, reduce or prevent that subject's conversion to a disease state. Similarly, identifying a subject's ASD severity enables the selection and initiation of various therapeutic treatment regimens to manage or reduce that subject's disease state. Monitoring the levels of at least one factor also allows for the course of treatment of ASD to be monitored. Such treatment regimens or therapeutic interventions can include exercise regimens, dietary modification, dietary supplementation, bariatric surgical intervention, administration of pharmaceuticals, communication therapy, behavioral therapy, occupational therapy, complementary medicine therapy, alternative medicine therapy, and treatment with therapeutics or prophylactics used in subjects diagnosed or identified with ASD. In one embodiment, the treatment may comprise intervention therapy, such as early intervention therapy and occupational therapy to improve communication, locomotion, and socialization ability in a subject less than 36 months of age.

The factors of the present invention can thus be used to generate a health profile or signature of subjects: (i) who do not have and are not expected to develop ASD and/or (ii) who have or expected to develop ASD. The health profile of a subject can be compared to a predetermined or reference profile to diagnose or identify subjects at risk for developing ASD, to monitor the progression of disease, as well as the rate of progression of disease, and to monitor the effectiveness of ASD treatments. Data concerning the factors of the present invention can also be combined or correlated with other data or test results, such as, without limitation, measurements of clinical parameters or other algorithms for ASD. In one embodiment, the health profile may contain a record of ED utilization by the subject.

The present invention also provides methods for identifying agents for treating ASD that are appropriate or otherwise customized for a specific subject. In this regard, a test sample from a subject, exposed to a therapeutic agent or a drug, can be taken and the type and quantity of the factor can be determined. The type and quantity of the factor can be compared to a profile before and after treatment, or can be compared to health profiles from one or more subjects who have shown improvements in risk factors as a result of such treatment or exposure.

In another embodiment, the invention is a method of monitoring the progression of ASD by assessing the factors of the invention.

Information obtained from the methods of the invention described herein can be used alone, or in combination with other information (e.g., disease status, disease history, vital signs, blood chemistry, ED utilization etc.) from the subject or from a biological sample obtained from the subject.

In other various embodiments of the methods of the invention, the level of one or more factors of the invention is determined to be increased when the level of one or more of the factors of the invention is increased by at least 10%, by at least 20%, by at least 30%, by at least 40%, by at least 50%, by at least 60%, by at least 70%, by at least 80%, by at least 90%, or by at least 100%, when compared to with a comparator control.

Various embodiments of the present invention describe mechanisms configured to monitor, track, and manage symptoms of ASD.

The system of the invention allows for monitoring comorbid conditions associated with ASD. In one embodiment, the system allows for the collection of data for the presence of comorbid conditions associated with ASD. The system can notify the user/evaluator about the likelihood of being as risk of developing ASD or having ASD. In another embodiment, the system can notify the user/evaluator about the severity of ASD and update the user/evaluator on changes in severity. For example, in some implementations, the system records the presence of a comorbid condition entered into the system by the user/evaluator or automatically recorded by the system and applies algorithms to recognize patterns that predict diagnosis and clinical features of ASD. The algorithmic analysis, for example, may be conducted in a central (e.g., cloud-based) system. Data uploaded to the cloud can be archived and collected, such that learning algorithms refine analysis based upon the collective data set of all patients. In some implementations, the system combines quantified clinical features and physiology to aid in diagnosing ASD objectively, early, and at least semi-automatically based upon collected data.

In some embodiment, the evaluator reviews a recorded session uploaded to the central system and makes a diagnosis. Evaluators, in some implementations, may perform a live (supervised) session, or review another clinician's live session, through real-time data feed between the family home session and a remote evaluator computing system. Although described as an in-home system due to the advantages described above, the system may additionally be used within a clinical environment to aid in evaluation of a subject.

The system has several advantages. Evaluation can be performed in the home, in a play-space, in the family's language, whenever the caregiver has time, discreetly, privately, rapidly, and inexpensively. The system can be in a form of a kit or an application in the context of an electronic device, such as an electronic hand held device or even a wearable data collection device for convenience. The system is beneficial to evaluators as well. Evaluators get access to many more subjects. The evaluators can perform diagnosis from home, during commute, or otherwise away from the office. Evaluators are afforded the opportunity to observe patients in their natural environment, and can witness transient behavioral events that otherwise only caregivers might see. If evaluators are working as a team to review an individual, they do not have to match their schedules to be in one place, but can jointly observe a single session with the individual from as many locations as there are team members. Above all, the system could decrease the age of ASD diagnosis drastically, and reach many at-risk families early.

In some implementations, the system goes beyond the evaluation stage to track an individual's ongoing progress. For example, after diagnosis of ASD, there is typically a long series of interventions from schools, doctors, etc. At some point, the child either does or does not develop socially and academically to a level where she can function in society. In between, there is rarely a point for interim evaluation and assessment to gauge progress. Maybe, a few years down the road, the family will have a follow-up “diagnosis” appointment. However, the follow-up visit will likely involve a different set of professionals, leaving the evaluation open to vagueness. Some programs for tracking ASD progress exist, having set goals and milestones, but they too can be vague and infrequently assessed. In employing a system such as the wearable data collection system described above for ongoing tracking of behaviors, abilities, and functionality of an ASD diagnosed child, a family can benefit from an exacting, quantitative-by-nature, cheap, at-home, understandable, standardized, comparable (from one time point to another), numerical assessment of a child's individual characteristics. The system, for example, could provide high-frequency (e.g., up to daily) assessments, each with perhaps hundreds or thousands or more data points or samples such as (in)correct behaviors or relevant brain states, which can be incorporated into the child's everyday home life to measure the child's ongoing progress.

To enable such ongoing assessment, in some embodiments, applications for assessment as the child's development progresses may be made available for download to or streaming on a wearable data collection device via a network-accessible content store other content repositories, or other content collections. Content providers, in some examples, can include educators, clinicians, physicians, and/or parents supplied with development abilities to build new modules for execution on the wearable data collection device evaluation and progress tracking system. Content can range in nature from simple text, images, or video content or the like, to fully elaborated software applications (“apps”) or app suites. Content can be freely available or subscription based. Content can be stand-alone, can be playable on a wearable data-collection device based on its existing capabilities to play content (such as in-built ability to display text, images, videos, apps, etc., and to collect data), or can be played or deployed within a content-enabling framework or platform application that is designed to incorporate content from content providers. Content consumers, furthermore, can include individuals diagnosed with ASD or their families as well as clinicians, physicians, and/or educators who wish to incorporate system modules into their professional practices.

In one embodiment, the system for assessing the degree of ASD of the invention can be implemented on a cell phone, tablet computer, a desk top computer, and the likes. In one embodiment, an evaluator such as a caretaker enters information for a specific child/patient and gets a message reflecting an assessment (e.g. go see a healthcare professional), and optionally may provide or direct the user to sources of educational materials.

In some implementations, in addition to assessment, one or more modules of the system provide training mechanisms for supporting the individual's coping and development with ASD and its characteristics such as, in some examples, training mechanisms to assist in recognition of emotional states of others, social eye contact, language learning, language use and motivation for instance in social contexts, identifying socially relevant events and acting on them appropriately, regulating vocalizations, regulating overt inappropriate behaviors and acting-out, regulating temper and mood, regulating stimming and similar behaviors, coping with sensory input and aversive sensory feelings such as overload, and among several other things, the learning of abstract categories.

In one embodiment, the system of the invention can be in a medium that operates automatically behind the scenes in an electronic medical records database/software so that a notice automatically occurs if the data is designated to prompt an alert.

In another embodiment, the system of the invention can be in a format that encompasses “machine learning” so the process and comparator are update and improved as more information is entered and new analogs are developed.

In one embodiment, the present invention relates to a system and methods for inexpensive, non-invasive measuring and monitoring comorbid conditions associated with ASD.

In one embodiment, the invention provides a system where the subject, preferably an early child, would be assessed for ASD in the family's regular home environment, on their schedule, in their language, and perhaps while allowing remote doctors to observe through the caregiver's eyes at the child (and vice versa). It should be private and confidential, quick and convenient, quantitative and repeatable, and low-cost enough that a worried parent will pay the cost directly (thus bypassing the complexity of insurance reimbursements).

### Treatments

In certain embodiments, treatment comprises administering a disease-modulating drug to a subject. The drug can be a therapeutic or prophylactic used in subjects diagnosed or identified with a disease or at risk of having the disease. In certain embodiments, modifying therapy refers to altering the duration, frequency or intensity of therapy, for example, altering dosage levels.

In various embodiments, effecting a therapy comprises causing a subject to or communicating to a subject the need to make a change in lifestyle, for example, increasing exercise, changing diet, and so on. The therapy can also include surgery.

Therapies that may be administered to a subject identified as having or being at risk of ASD may include, but are not limited to, early intervention therapy, pivotal response treatment (PRT), verbal behavior (VB) therapy, applied behavioral analysis (ABA), discrete trial teaching (DTT), floortime (aka. developmental individual difference relationship model (DIR)), relationship development intervention (RDI), training and education of autistic and related communication handicapped children (TEACCH), social communication/emotional regulation/transactional support (SCERTS), speech-language therapy (SLT), occupational therapy (OT), sensory integration (SI), physical therapy (PT), social skills therapy, picture exchange communication system (PECS), auditory integration therapy (AIT), gluten free casein free diet (GFCF), early start Denver model (ESDM), behavioral therapy, communication therapy, dietary therapy, drug therapy, complementary medicine therapy, alternative medicine therapy or any combination thereof.

Assessment of the factors of the invention allow for the course of treatment of a disease to be monitored. The effectiveness of a treatment regimen for a disease can be monitored by detecting one or more factors from a subject over time and comparing the type and quantity of factors detected. For example, a health profile can be obtained prior to the subject receiving treatment and one or more subsequent health profiles are taken after or during treatment of the subject. Changes in the health profile may provide an indication as to the effectiveness of the therapy.

In various exemplary embodiments, effecting a therapy comprises administering a disease-modulating drug to the subject. The subject may be treated with one or more disease-modulating drugs until the factors or health profile return to a baseline value measured in a population not suffering from the disease, experiencing a less severe stage or form of a disease or showing improvements as a result of treatment with a disease-modulating drug.

A number of compounds such as a disease-modulating drug may be used to treat a subject and to monitor progress using the methods of the invention. The beneficial effects of these and other drugs can be visualized by assessment of the factors of the invention.

Any drug or combination of drugs may be administered to a subject to treat a disease. The drugs herein can be formulated in any number of ways, often according to various known formulations in the art or as disclosed or referenced herein.

In various embodiments, any drug or combination of drugs disclosed herein is not administered to a subject to treat a disease. In these embodiments, the practitioner may refrain from administering the drug or combination of drugs, may recommend that the subject not be administered the drug or combination of drugs or may prevent the subject from being administered the drug or combination of drugs.

In various embodiments, one or more additional drugs may be optionally administered in addition to those that are recommended or have been administered. An additional drug will typically not be any drug that is not recommended or that should be avoided. In exemplary embodiments, one or more additional drugs comprise one or more glucose lowering drugs.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Identifying Comorbid Conditions as Early Indicators of ASD

A sample size of at least 100,000 children with medical records from birth up to 5 years old was used to identify comorbid conditions.

Data source

MarketScam Commercial Claims and Encounters (CCE) database comprising of health care claims for employees and their dependents of over 250 medium and large employees nationwide was used.

### Study Design

A retrospective cohort study was performed to identify comorbidities associated with ASD. ICD9 codes and CPT4 codes were used to identify disease diagnosis, procedures, and lab tests for each individual. The study cohort consisted of a longitudinal cohort of ASD children who were diagnosed with ASD during the study period and non-ASD children.

### Study Cohort Summary

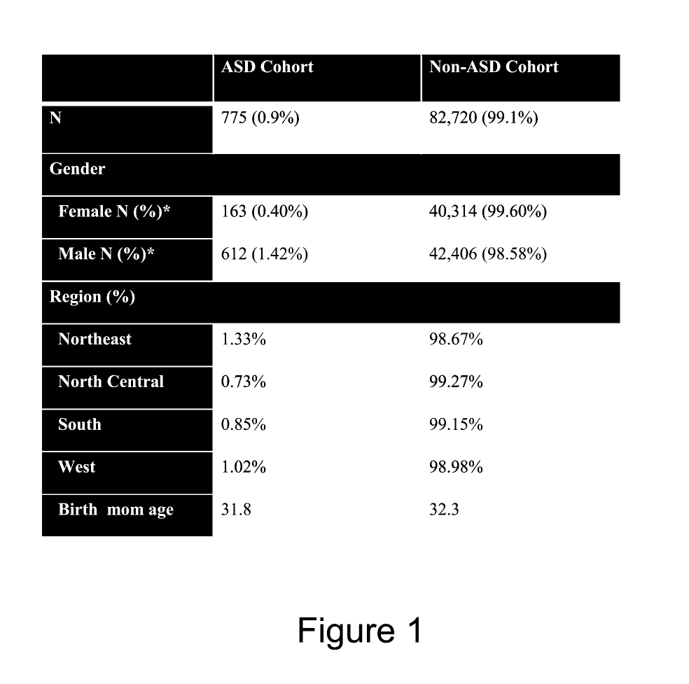

An overview of the study cohort is provided in FIG. 1. A total of 775 children were identified as the ASD sub-cohort based on a diagnoses of ASD (ICD 9 codes 299.0x and 299.8x). The non-ASD sub-cohort of children consisted of those individuals without an ASD diagnosis. In each of the sub-cohorts, adolescents were further categorized by gender, US census regions (i.e. Northeast, North central, South, and West), and the age of the birth mother.

### Statistical Analysis

A dichotomous outcome was defined as ASD vs. Non-ASD. A leveled outcome was defined in terms of the severity of ASD. Descriptive analyses were used for exploring cohort demographic information as appropriate. Univariate tests (chi-squares for categorical variables; Student t-test for continues variables) were used to assess the associations between ASD and each of the factors of interest. Logistic regression and generalized linear model (GLM) were used for multivariable analysis.

### Results

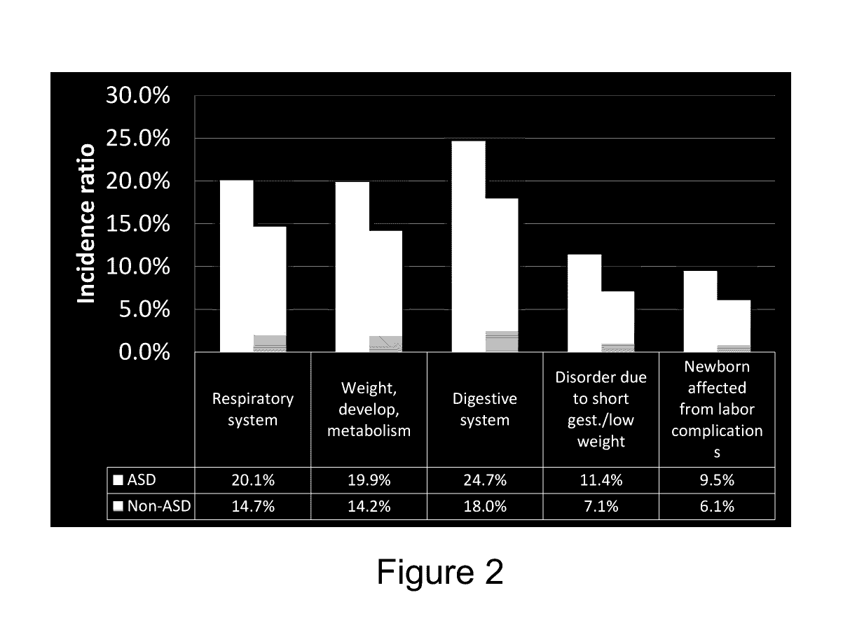

It was observed that during the 1^{st} 180 days since birth, ASD children had significantly higher odds than non-ASD children (all p-values<0.0001) of having diseases in respiratory system and digestive system, delay in gaining weight and reaching other developmental milestones; disorders due to short gestation and low birth weight; and problems due to complications during labor (FIG. 2).

A more detailed analysis of the 5 categories from FIG. 2 shows the following: 22.71% of autistic kids had at least 2 of those 5 categories in FIG. 2 vs. 12.96% from the non-autism cohort; 5.76% (ASD) vs. 1.44% (non-ASD) had unspecified hearing loss (ICD 9 code 389.9); 5.18% (ASD) vs. 0.51% (non-ASD) had lack of coordination (ICD 9 code 781.3); 5.90% (ASD) vs. 3.90% (non-ASD) had influenza with other manifestation (ICD 9 code 389.9); 2.59% (ASD) vs. 1.42% (non-ASD) had patent ductus arteriosus (ICD 9 code 747.0); 3.45% (ASD) vs. 0.55% (non-ASD) had dysphagia (ICD 9 code 787.20); 6.04% (ASD) vs. 3.80% (non-ASD) had abdominal pain (ICD 9 code 789.00); 4.75% (ASD) vs. 1.17% (non-ASD) had other convulsion (ICD 9 code 780.39); 9.06% (ASD) vs. 5.38% (non-ASD) had ear pain (ICD 9 code 388.70); 9.93% (ASD) vs. 6.94% (non-ASD) had teething syndrome (ICD 9 code 520.7); 6.62% (ASD) vs. 0.63% (non-ASD) had delayed milestone (ICD 9 code 783.42); 6.47% (ASD) vs. 4.24% (non-ASD) had other respiratory abnormalities (ICD 9 code 786.09); 7.05% (ASD) vs. 4.77% (non-ASD) had asthma, unspecified type (ICD 9 code 493.90); 6.33% (ASD) vs. 0.55% (non-ASD) had unspecified delay in development (ICD 9 code 315.9); 5.32% (ASD) vs. 3.28% (non-ASD) had other preterm infants, unspecified [weight] (ICD 9 code 765.10); 11.65% (ASD) vs. 7.62% (non-ASD) had rash and other non-specified skin eruption (ICD 9 code 782.1); 8.06% (ASD) vs. 5.47% (non-ASD) had constipation, unspecified (ICD 9 code 564.00); 9.64% (ASD) vs. 6.63% (non-ASD) had other convulsion (ICD 9 code 786.07); 9.50% (ASD) vs. 7.63% (non-ASD) had diarrhea (ICD 9 code 787.91); 13.96% (ASD) vs. 10.33% (non-ASD) had contact dermatitis and other eczema (ICD 9 code 692.9); 11.22% (ASD) vs. 7.84% (non-ASD) had other atopic dermatitis and related conditions (ICD 9 code 691.8); 8.06% (ASD) vs. 1.34% (non-ASD) had lack of normal physiological development, unspecified (ICD 9 code 783.40); 10.36% (ASD) vs. 6.97% (non-ASD) had vomiting alone (ICD 9 code 787.03); 11.22% (ASD) vs. 8.83% (non-ASD) had acute laryngitis and trachetitis (ICD 9 code 464.4); 10.94% (ASD) vs. 6.96% (non-ASD) had feeding difficulty (ICD 9 code 783.3); 12.66% (ASD) vs. 7.68% (non-ASD) had colitis (ICD 9 code 558.9); 28.20% (ASD) vs. 19.05% (non-ASD) had unspecified viral infection (ICD 9 code 079.99); 17.84% (ASD) vs. 12.80% (non-ASD) had acute pharyngitis (ICD 9 code 462); 22.73% (ASD) vs. 17.48% (non-ASD) had cough (ICD 9 code 786.2); 17.97% (ASD) vs. 11.27% (non-ASD) had GERD (ICD 9 code 530.81); 27.91% (ASD) vs. 21.34% (non-ASD) had fever (ICD 9 code 780.60); 34.96% (ASD) vs. 28.94% (non-ASD) had Otitis Media (ICD 9 code 382.9); 56.83% (ASD) vs. 48.52% (non-ASD) had acute upper respiratory infection (ICD 9 code 465.9).

The results indicate that the ASD children have a distinct medical profile in early childhood, well before the onset of typical ASD-specific behavior signatures. As the evidence suggests, ASD can start as early as the second trimester, brain and other physiological changes due to ASD may leave the child vulnerable to certain diseases at early ages well before the manifestation of typical ASD symptoms.

The results presented herein demonstrate a proof of principle of an establishment of a risk index for stratification of ASD risks by integrating the already known ASD risk factors with the newly ASD risk factors presented herein and development of a prediction model for personalized probabilistic assessment of ASD risks.

### Example 2

### A Profile on Emergency Department Utilization in Adolescents and Young Adults with Autism Spectrum Disorder

As individuals with ASD age, it has been found that physical health service needs are unmet and that those services individual do have are unsatisfactory (Bureau of Autism Services 2011). Barriers to accessing care was reported to be cost and insurance, availability of providers who will provide healthcare to adolescents and young adults with ASD, as well as transportation barriers when living in rural areas (Bureau of Autism Services 2011; Thomas et al., J Autism Dev Disord. 2007, 37:1902-1912). However, while there is a reported unmet need for services, concurrently research in the past decade has consistently documented a higher rates of healthcare utilization in individuals with ASD (Boulet et al., Arch Pediatr Adolesc Med. 2009, 163:19-26; Croen et al., Pediatrics, 2006, 118:e1203-e1211; Kogan et al., Pediatrics, 2008, 122:e1149-e1158; Warfield and Gulley, Matern Child Health J. 2006, 10:201-216; Liptak et al., J Autism Dev Disord. 2006, 36:871-879). More specifically, Leonard and colleagues (Leonard et al., Soc Sci Med. 2005, 60:1499-1513) found that during inpatient hospitalizations, irrespective of the reason for admission, children with ASD had an increased rate of contact during their hospital stay.

The cost of medical care for children and adolescents with ASD have been found to be approximately 3 to 7 times greater than those individuals without ASD (Croen et al., Pediatrics, 2006, 118:e1203-e1211; Peacock et al. J Dev Behav Pediatr., 2010, 33:2-8; Shimabukuro et al., J Autism Dev Disord., 2008, 38:546-552). This increased cost is likely associated with increased lengths of hospitalizations found for children and adolescents with ASD as compared to individuals without ASD (Kato et al., Gen Hosp Psychiatry. 2013, 35:50-53; Lokhandwala et al., J Autism Dev Disord. 2012, 42:95-104). More concerning is that as children with ASD age into adolescence and adulthood, the cost of healthcare increases (Croen et al., Pediatrics, 2006, 118:e1203-e1211; Leslie and Martin, Arch Pediatr Adolesc Med. 2007, 161:350-355; Newacheck and Kim, Arch Pediatr Adolesc Med. 2005, 159:10-17). This cost has been found to be particularly high for adolescents with ASD between the ages of 15- and 18-years-old and has been posited to result from increased prescription medication use and inpatient hospitalizations (Croen et al., Pediatrics, 2006, 118:e1203-e1211; Shimabukuro et al., J Autism Dev Disord., 2008, 38:546-552). Children and adolescents with ASD and non-psychiatric (e.g., epilepsy) and/or psychiatric comorbidities (e.g., ID, mood disorder) have been found to have higher health care costs (Croen et al., Pediatrics, 2006, 118:e1203-e1211; Peacock et al. J Dev Behav Pediatr., 2010, 33:2-8). More specifically, it was found that children under 10-years-old had a larger number of non-psychiatric health care costs, while children and adolescents over the age of 10-years-old had a larger number of psychiatric health care costs.

When examining psychiatric comorbidities, the cost remained higher even in comparison to age-matched individuals with similar psychiatric diagnoses without ASD (Croen et al., Pediatrics, 2006, 118:e1203-e1211; Peacock et al., J Dev Behav Pediatr., 2010, 33:2-8). Many researchers have found that children and adolescents with ASD are at greater risk for psychiatric hospitalization than children with other disorders (Bebbington et al., BMJ Open, 2013, 3:1-10; Gallaher et al., Arch Pediatr Adolesc Med. 2002, 156:246-251; Mandell, Pediatrics, 2008, 38:1059-1069; Saeed et al., J Behav Health Sery Res. 2003, 30:406-417). Mandell found that before reaching 21-years-old approximately 10% of children and adolescents with ASD are admitted to the hospital as a result of a psychiatric crisis (Mandell, Pediatrics, 2008, 38:1059-1069). More concerning, Mandell also found that the risk of hospitalization was increasing over time (Mandell,

Pediatrics, 2008, 38:1059-1069). Although none would argue that there are not cases where hospitalization is justified for children and adolescents with ASD, it is likely that the current rates of healthcare utilization and cost for this population is inflated and may reflect a lack of lower-level care and services (Green et al. J Am Acad Child Adolesc Psychiatry, 2001, 40:325-332; Leichtman et al. Am J Orthopsychiatry, 2001, 71:227-235).

### Emergency Department (ED) Utilization

There has been a steady increase in utilization of the ED for both non-psychiatric and psychiatric referrals by children and adolescents over time (McCraig and Burt, National Hospital Ambulatory Medical Care Survey: 2003 emergency department summary. 2005, 358; Mahajan et al., Pediatr Emerg Care, 2009, 25:715-720). This trend has also been documented in children and adolescents with ASD and has been found to be increasing at an even higher rate when compared to individuals without ASD (Gurney et al., Arch Pediatr Adolesc Med., 2006, 160:825-830). Deavenport-Saman and colleagues found that children and adolescents with ASD had 0.26 more documented visits to the ED when compared to individuals without ASD (Deavenport-Saman et al., Matern Child Health J. 2016, 20:306-314). Moreover, they found that children specifically who presented to the ED were more likely to be older (i.e., school-age compared to preschool-age). A recent examination of ED utilization in adults with ASD found a similar trend in high utilization of the ED (Nicoliadis et al., J Gen Intern Med. 2013, 28:761-769).

### Reasons for Referral to ED

Recent research has examined the common presenting issue for children and adolescents with ASD when arriving at the ED, including both non-psychiatric and psychiatric issues. The most common non-psychiatric presenting problems include epilepsy, seizures, and/or neurological symptoms (9-15%) and gastrointestinal disturbances (15%) such as nausea, vomiting, diarrhea, abdominal pain, and constipation (Buie et al., Pediatrics, 2010, 125:S1-S18; Cohen-Silver et al., Clin Pediatr (Phila). 2014, 53:1134-1138; Coury, Curr Opin Neurol. 2009, 23:131-136; Deavenport-Saman et al., Matern Child Health J. 2016, 20:306-314; Tuchman et al., Brain Dev. 2010, 32:719-730; Vohra et al., J Autism Dev Disord. 2016, 46:1441-1454; Wang et al., Appl Environ Microbiol. 2011, 77:6718-6721). In their sample, Deavenport-Saman and colleagues found that 7% of children and adolescents with ASD presented to the ED with upper respiratory infections, 7% presented with viral infections, and 5% presented with otitis media. Previous research has found that children and adolescents with ASD have had an increased relative risk for injury although only 4% of these individuals presented to the ED with unspecified head injuries and 3% presented with dental injuries (Deavenport-Saman et al., Matern Child Health J. 2016, 20:306-314; McDermott et al., J Autism Dev Disord. 2008, 38:626-633).

Sills and Bland found that psychiatric issues were the presenting issue in 1.6% of all ED visits for children and adolescents in general (Sills and Bland, Pediatrics, 2002, 110:e40). Since that time, psychiatric-related ED visits have been found to be increasing at a swifter rate than non-psychiatric reasons for referral to the ED (Larkin et al., Psychiatr Serv. 2005, 56:671-677). Additionally, Kalb and colleagues found that ED visits for children and adolescents with ASD were more likely to be related to a psychiatric concern then non-psychiatric concern (Kalb et al., Pediatr Emerg Care. 2012, 28:1269-1276). In regards to common reasons for referral to the ED that are psychiatric in nature, Iannuzzi and colleagues found that as children with ASD entered elementary school behavioral issues became a common reason for referral to the ED (Iannuzzi et al., J Autism Dev Disord. 2015, 45:1096-1102). However, around the time of entry into middle school, in addition to behavioral issues mood symptoms, self-injurious behavior, and more significant aggression entered into the top reasons for referral to the ED and remained stable into young adulthood (Iannuzzi et al., J Autism Dev Disord. 2015, 45:1096-1102). Finally, Wharff and colleagues found that children with developmental disabilities, including ASD, were 2.5 times more likely to utilize the ED while waiting for an opening on a psychiatric inpatient facility (Wharff et al., Pediatr Emerg Care. 2011, 27:483-489).

Approximately one-fourth of children and adolescents with ASD have been found to make repeated visits to the ED and of this 25%, half of the individuals had been to the ED in the two weeks prior to the current visit (Cohen-Silver et al., Clin Pediatr (Phila). 2014, 53:1134-1138). Of the children and adolescents with ASD who present to the ED, almost 20% have been found to have been subsequently admitted to the hospital compared to a rate of 10% in children and adolescents without ASD (Cohen-Silver et al., Clin Pediatr (Phila). 2014, 53:1134-1138). Deavenport-Saman and colleagues found that children and adolescents with ASD were less likely to be admitted to the hospital from the ED if they arrived at the ED during weekday daytime or evening hours, were female, were English-speaking, and who had no insurance. In the same sample, being 6-years-old or older, being non-Hispanic, and traveling a greater distance to the ED led to high admittance rates (Deavenport-Saman et al., Matern Child Health J. 2016), 20:306-314. Finally, in a sample of adults with ASD, Vohra and colleagues found that approximately 33% of ED visits led to a hospital admission while only 10% of adults without ASD were admitted to the hospital after an ED visit (Vohra et al., J Autism Dev Disord. 2016, 46:1441-1454).

Children in rural areas have been found to have less access to regular and specialty medical and mental health care and therefore may be more likely to present at the Emergency Department for mental health care (Cohen and Hesselbart, Am J Public Health. 1993, 83:49-52; Slade, J Behav Health Sery Res. 2003, 30:382-392; Thomas et al., J Autism Dev Disord. 2007, 37:1902-1912). Approximately half of families of children with ASD living in a rural community reported that they experienced problems with the services they received, most often as a result of lack of availability of well-trained providers (Hutton et al., Focus Autism Other Dev Disabl. 2005, 20:180-189). Dew and colleagues documented that caregivers of individuals with ASD in rural communities in Australia noted a lack of ASD expertise in healthcare providers in rural and remote areas of the country (Dew et al., Health Soc Care Community, 2013, 21:432-441). Thomas and colleagues found that caregivers of children with ASD who lived in rural communities reported significantly less access to special summer camps and respite care (Thomas et al., J Autism Dev Disord. 2007, 37:1902-1912). Another indicator of a paucity of services in rural communities is the documented older mean age of diagnosis of ASD for individuals living in a rural community when compared to those in suburban or urban settings (Mandell et al., Pediatrics, 2005, 116:1480-1486). Recent research has begun to examine the efficacy of telehealth remote technology, group-based parent interventions, and training models for PCPs to meet the needs of individuals with ASD and their families in rural settings (Farmer and Reupert, Aust J Rural Health. 2013, 21:20-27; Meadan et al., Rural Special Education Quarterly, 2013, 32:3-10).

Several predictors of ED utilization have been examined in children and adolescents with ASD. Day and time has been found to predict increased ED utilization in children and adolescents with ASD. Recent research has found that the majority of children and adolescents with ASD present to the ED during weekday, daytime hours (Cohen-Silver et al., Clin Pediatr (Phila). 2014, 53:1134-1138). This was increased utilization was found to hold steady in comparison to children and adolescents without ASD's utilization of the ED (Deavenport-Saman et al., Matern Child Health J. 2016, 20:306-314). When examining type of insurance and the ability to predict ED utilization, the results have been mixed. Deavenport-Saman and colleagues found that children and adolescents with ASD who presented to the ED were more likely to have public insurance (i.e., Medicare managed care or Medicaid fee-for-service) in comparison to typically developing youth (Deavenport-Saman et al., Matern Child Health J. 2016, 20:306-314). On the other hand, Kalb and colleagues found that children and adolescents with ASD who had private insurance had an increased likelihood of an ED visit for psychiatric reasons when compared to children and adolescents without ASD (Kalb et al., Pediatr Emerg Care. 2012, 28:1269-1276).

Although there has been substantial research on the medical and psychiatric complexities in children and adolescents with ASD, recent research suggests that the minority of ED visits are warranted for a high level of care. Cohen-Silver and colleagues found that only 44% of ED visits in children and adolescents with ASD were a result of a presenting complaints defined as high acuity and children and adolescents with ASD were 2.6% more likely to have non-urgent ED visits compared to those without ASD (Cohen-Silver et al., Clin Pediatr (Phila). 2014, 53:1134-1138). Similarly, Soto and colleagues reported in their sample that more than one-third of children and adolescents with ASD referred to the ED could be handled in a “less restrictive environment” (Soto et al., J Clin Psychiatry. 2009, 70:1164-1177)

Many researchers have proposed that the increased ED utilization for children and adolescents with ASD may be related to the deficit in first line, community-based, outpatient care (Green et al. 2001; Leichtman et al. 2001). Children and adolescents with ASD have been documented as having significant difficulty accessing specialty medical and mental health care when compared with other youth with disabilities (Krauss et al., Ment Retard. 2003, 41:329-339; Soto et al., J Clin Psychiatry. 2009, 70:1164-1177). Soto and colleagues found that of children and adolescents with ASD who present to the ED, approximately two-thirds current reported that they had ongoing outpatient care, but utilized this infrequently (Soto et al., J Clin Psychiatry. 2009, 70:1164-1177).

Nageswaran and colleagues found that parents of children and adolescents with ASD report decreased satisfaction with their children's general health care (Nageswaran et al., Matern Child Health J. 2011, 15:634-641). Caregivers of children and adolescents with ASD have reported that they have significant trouble accessing their PCP which has been proposed to lead to increased ED visits and hospitalization rates higher than children and adolescents without ASD (Bebbington et al., BMJ Open, 2013, 3:1-10, Krauss et al., Ment Retard. 2003, 41:329-339). Gurney and colleagues found that caregivers of children and adolescents with ASD reported that they believe their child's PCP does not have a salient role in the care of their child's health (Gurney et al., Arch Pediatr Adolesc Med., 2006, 160:825-830). However, it was also posited that a key reason for an increase in ED visits by children and adolescents with ASD was the last of psychiatric evaluations available in outpatient mental health clinics and educational settings (Soto et al., J Clin Psychiatry. 2009, 70:1164-1177).

Caregivers of typically developing children and adolescents who reported that their PCP stresses family centeredness, timeliness, and coordinated care have been found to report decreased numbers of visits to the ED (Brousseau et al., Acad Pediatr. 2009, 9:33-39; Piehl et al., Arch Pediatr Adolesc Med. 2000, 154:791-795; Wang et al., Pediatrics, 2005, 116:1075-1079). Similarly, caregivers of children and adolescents with ASD report they are more likely to access the ED for healthcare when they perceived their healthcare providers do not listen to their concerns, display cultural insensitivity, do not supply needed information, and do not involve caregivers in decision making (Lin et al., Pediatr Emerg Care. 2014, 30:534-539). Therefore, it is likely that caregivers of youth with ASD access their PCPs for routine visits that are for minor issues as a result of having less assurance for help with acute, emergent, and/or complex behavioral or health issues (Kogan et al., Pediatrics, 2008, 122:e1149-e1158).

There have been several studies since 2009 that have examined ED utilization in a pediatric sample of individuals with ASD (Cohen-Silver et al., Clin Pediatr (Phila). 2014, 53:1134-1138; Kalb et al., Pediatr Emerg Care. 2012, 28:1269-1276; Mahajan et al., Pediatr Emerg Care, 2009, 25:715-720). The majority of these studies were descriptive in nature outlining the demographic characteristics and common presenting complaints of ED visits in children and adolescents with ASD (Iannuzzi et al., J Autism Dev Disord. 2015, 45:1096-1102; Kalb et al., Pediatr Emerg Care. 2012, 28:1269-1276; Mahajan et al., Pediatr Emerg Care, 2009, 25:715-720; Wu et al., Res Dev Disabil. 2014, 36:78-86) and in adults with ASD (Vohra et al., J Autism Dev Disord. 2016, 46:1441-1454). However, only more recently have researchers examined predictors and outcomes of children and adolescents with ASD (Cohen-Silver et al., Clin Pediatr (Phila). 2014, 53:1134-1138; Deavenport-Saman et al., Matern Child Health J. 2016, 20:306-314; Lunsky et al., Emerg Med J. 2015, 32:787-792). For example, in a survey of caregivers of individuals between the ages of 12- and 56-years-old, Lunsky and colleagues found that ED visits in the previous calendar year, a history of physical aggression toward others, and no structured daytime activities predicted higher rates of ED utilization in individuals with ASD (Lunsky et al., Emerg Med J. 2015, 32:787-792). These recent studies have utilized caregiver-report data of 396 adolescents and adults with ASD (Lunsky et al., Emerg Med J. 2015, 32:787-792), small-scale retrospective chart review (N=160; Cohen-Silver et al., Clin Pediatr (Phila). 2014, 53:1134-1138)), and a small timeframe (three years) retrospective analysis of ED discharge data (N=1,424 children with ASD; Deavenport-Saman et al., Matern Child Health J. 2016, 20:306-314).

The current study intends to examine the rates of ED utilization over eight years (2005-2013) in youth ages 12- to 21-years-old with ASD. Without being bound by any particular theory, it is hypothesized that 1) adolescents with ASD will have significantly more ED visits as compared to adolescents without ASD; 2) adolescents who are older (over the age of 14-years-old) will have significantly more ED visits as compared to younger adolescents (under the age of 14-years-old); and 3) adolescents living in rural communities will have significantly more ED visits as compared to younger adolescents (under the age of 14-years-old).

### The Materials and Methods are now Described

### Data Source

The MarketScan® Commercial Claims and Encounters database (Truven Health Analytics) consists of reimbursed healthcare claims from a selection of large employers and health plans. Included individuals are covered by private insurance plans across the United States (US), with claims information from more than 130 payers describing the healthcare use and expenditures for more than 50-million employees and family members per year. Ages range from birth to 65 years old when most individuals switch from private insurance to Medicare. Claims for individuals are identified by a unique patient identifier and contain information on inpatient, outpatient and prescription drug service use, as well as patient age, gender, geographic location, and type of health insurance plan. The medical claims contain medical diagnoses coded by the International Classification of Disease: Ninth Revision (ICD-9), whereas medical procedures are coded by Current Procedural Terminology, 4^{th} edition (CPT-4).

Study populations

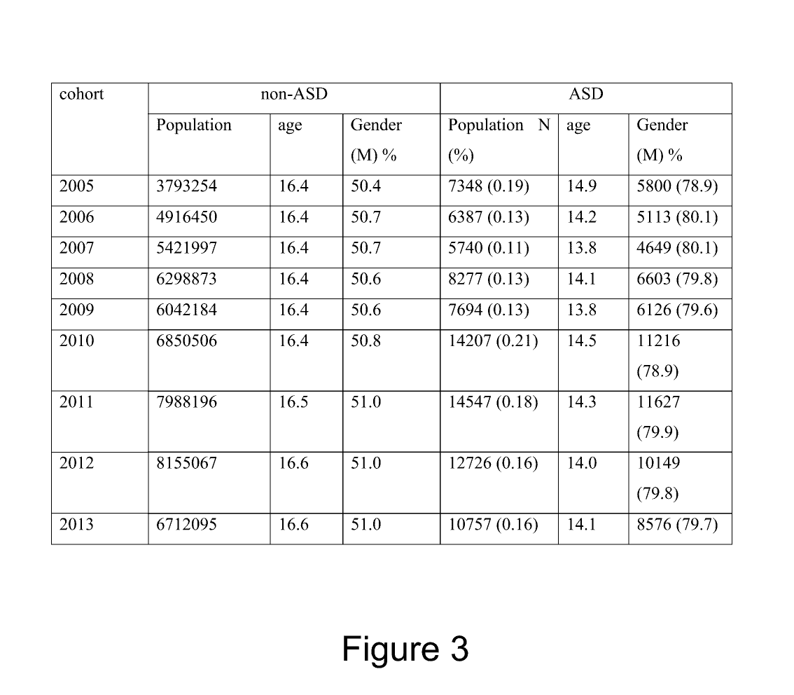

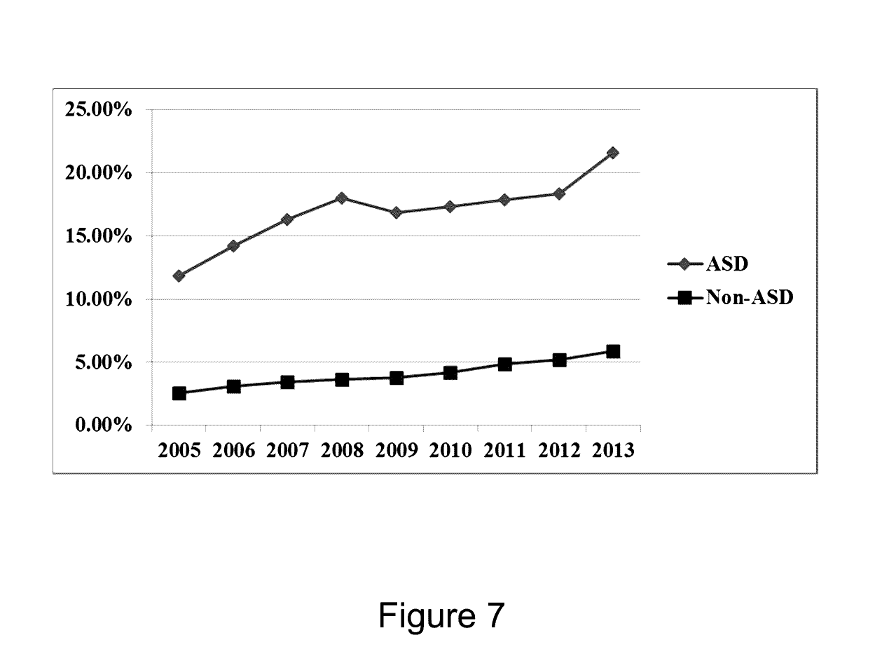

Using the healthcare claims data from MarketScan® between 2005 and 2013, a total of 56,266,305 individuals were identified between the ages 12- to 21-years-old in the current database (see FIG. 3). In each annual cohort, an ASD sub-cohort of adolescents was constructed of individuals with at least two separate diagnoses of ASD (ICD 9 codes 299.0x and 299.8x) through the entire study period (2005-2013; N=87,683) and a non-ASD sub-cohort of adolescents without ASD diagnosis (N=56,178,622) during the study period. In each of the annual cohort, adolescents were further categorized into three age brackets: early adolescence (12- to 14-years-old), middle adolescence (15- to 17-years-old) and older adolescence (18- to 21-years-old). Additionally, gender, US census regions (i.e. Northeast, North central, South, and West), type of residence (i.e., urban, rural), and type of health plans were documented. Type of residence was defined as residence within (urban) or outside of (rural) a metropolitan statistical area (msa). A non-zero msa code indicated an urban residence; while a rural residence indicated otherwise (i.e. rural). Nine types of health plans (i.e., basic/major medical, CDHP, comprehensive, EPO, HDHP, HMO, POS, POS with capitation, PPO, PPO, HMO) accounted for over 80% of the enrollees in the study cohort. Therefore, type of health plan was categories as PPO, HMO or a bundle of the remaining seven health plan types together. A behavioral service related ED visits was identified determined by an ED visit accompanied by a behavioral diagnosis (ICD 9 code 290-319) or behavioral service/consultation (ICD code V11, V40, V61, V62, V79). An ED visit without any behavioral service was considered a non-behavioral ED visit (FIG. 7).

Data analysis

By linking the in-patient and out-patient encounter claims database from 2005 to 2013, the annual utilization of the ED was estimated on an individual patient basis. The number of unique patients who had ED visit(s) among the cohort of adolescent with ASD and those without was calculated. In particular, the proportion of patients who visited ED among ASD vs. non-ASD adolescent cohorts was determined. As noted above, individuated were categorized by age bracket (i.e., early, middle, or late adolescence), gender, geographic location (i.e., urban, rural), type of health plan, and type of ED visit (i.e., behavioral, non-behavioral).

Descriptive analyses were used for exploring cohort demographics and the distributions of variables. Univariate association between each factor of interest and the outcome variable was examined using Chi-squared test/Fisher's exact test for categorical variables and t-test/Wilcoxon rank sum test for continuous variables. Multivariable analysis was performed by Logistic regression model. All statistical analyses were performed using SAS version 9.3 software (SAS Institute, Cary, NC). All statistical tests were two-sided, with p-values less than 0.05 being considered statistically significant.

### The Experimental Results are now Described

Despite the fact that there has been a widespread documentation of a steady increase of ASD prevalence since 2000, 1 in 68 in the 2014 CDC report, the data presented here does not show a monotonous increase in ASD prevalence among the target adolescent population in the ED. ASD prevalence was found to fluctuate between 0.11% and 0.21% from 2005 to 2013 and was 0.16% during 2013. This was consistent with the consensus that the increase of ASD prevalence was primarily from an increase of ASD cases early childhood and is a reflection of increased early screening/diagnosis, changes in diagnostic criteria and improved awareness of ASD population in children (Matson and Kozlowski, Res Autism Spectr Disord. 2011, 5:418-425). The lower ASD prevalence rates in this sample could also be reflective of under-coding by primary care physicians and lack of access to specialized services, such as behavioral health services, where an ASD diagnosis may be more likely to be documented.

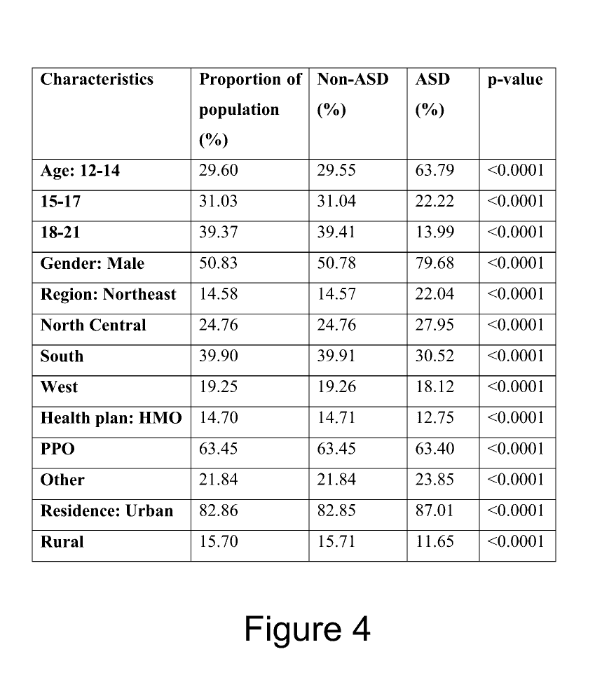

Consistent with previous findings in the literature, the majority of the ASD patients in the study cohort were male; the overall ratio was approximately 4:1 male to female. The gender ratio for the entire cohort, which included individuals with and without ASD, was 51:49 male to female. The ASD adolescent population presenting to the ED was younger (approximately 14-years-old) than the non-ASD population (approximately 16) in the annual cohorts. In particular, over 60% of the ASD cohort was just entering the early adolescent group, 22% fell in the middle adolescence group, and only 14% fell in the late adolescence group. In contrast, the three age groups were more evenly represented in the non-ASD population, with early, middle, and late adolescence accounting for 30%, 31% and 39% of the non-ASD cohort (FIG. 4). The South US census region accounted for 40% of the total study population, while Northeast represented the lowest population in the cohort with 15%. PPO was the most used health plan (64%), followed by HMO (15%), while the remainder of the population was insured by the other seven smaller types of health plans. A majority of the study population (83%) lived closed to the metropolitan area (urban), defined by a non-zero metropolitan statistical area (msa) code; while the remainder of the population (17%) was defined as living in a non-metropolitan area (rural).

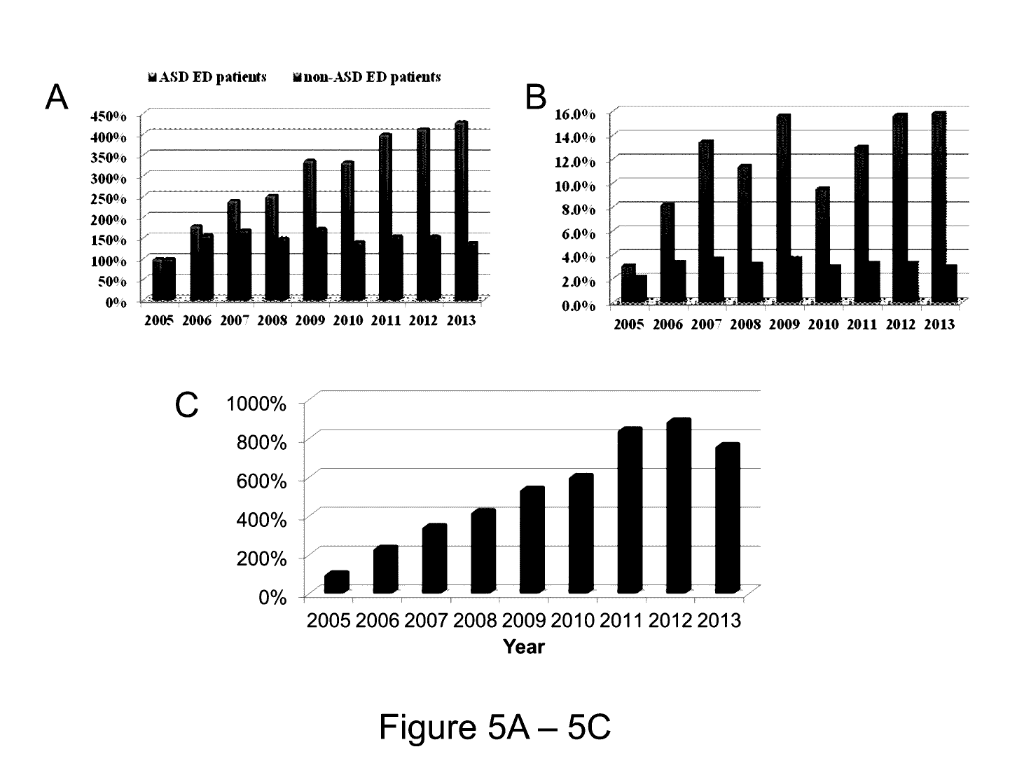

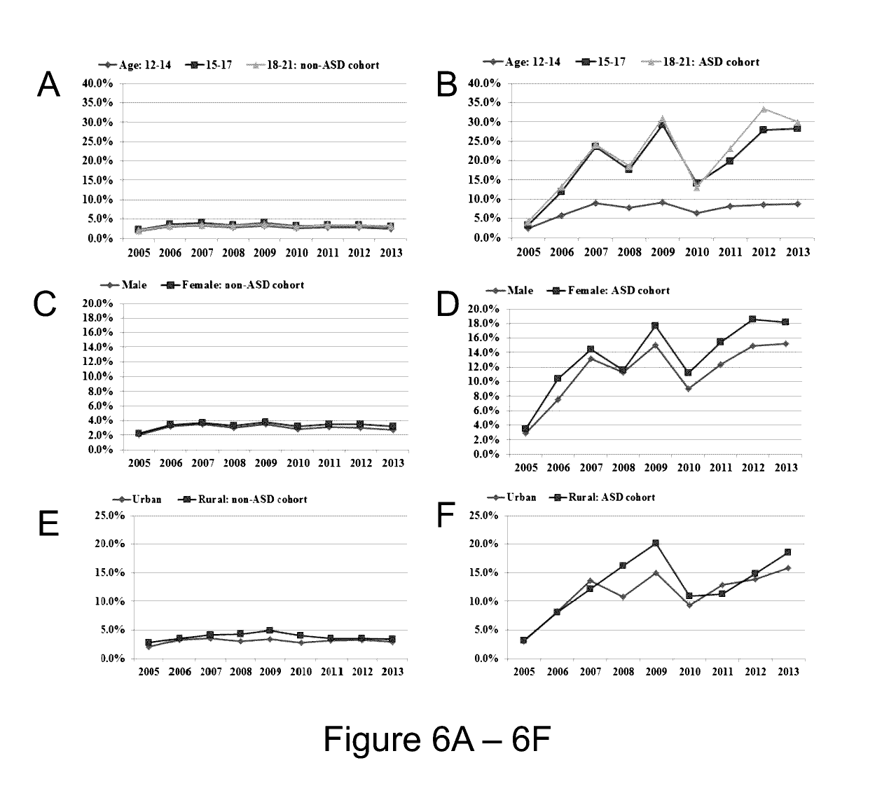

A consistent increase in the percentage of ASD patients among all adolescents who visited ED was observed, from 0.28% during 2005 to 0.85% by 2013. While the percentage of non-ASD patients who had ED visits have been fairly flat at around 3%, the percentage of ASD cohort patients who had ED visits steadily increased from 3% during 2005 to 16% by 2013, a five-fold increase from 2005 to 2013 equivalent to an annual 20% sequential increase (FIG. 5). A further breakdown (FIG. 6) by age group showed that, through the study period (2005-2013) there had been relatively smaller portion (less than 10%) early adolescents who had documented ED visits per year. However, a much larger proportion (30%) of the middle and late adolescent groups had documented ED visits. In contrast, only 3 to 4% of middle and late adolescent groups without ASD had documented ED visits.

While it was more likely that adolescents with ED visits were male, which is likely an artifact of the higher ASD prevalence among males, percentage-wise a larger of portion females than males had ED visits. A similar consistent pattern was observed, although at a much smaller scale, in the cohort without ASD; a higher percentage of females had ED visits than males. Furthermore, there was a similar, annual sequential increase of ED visits between male and female adolescents with ASD at 20%. Although rural adolescents with ASD showed a similar pattern as those living in urban areas, there was an observed increased from 15% during 2012 to 19% in 2013, compared to 16% among urban adolescents with ASD; and 3.0% of adolescents without ASD who lived in rural and urban areas, respectively. Among adolescents with ASD who had a documented

ED visit, those who had behavioral health service-related ED visits increased from 12% during 2005 to 22% by 2013, compared to an increase from 3% to 6% among adolescents without ASD (FIG. 7).

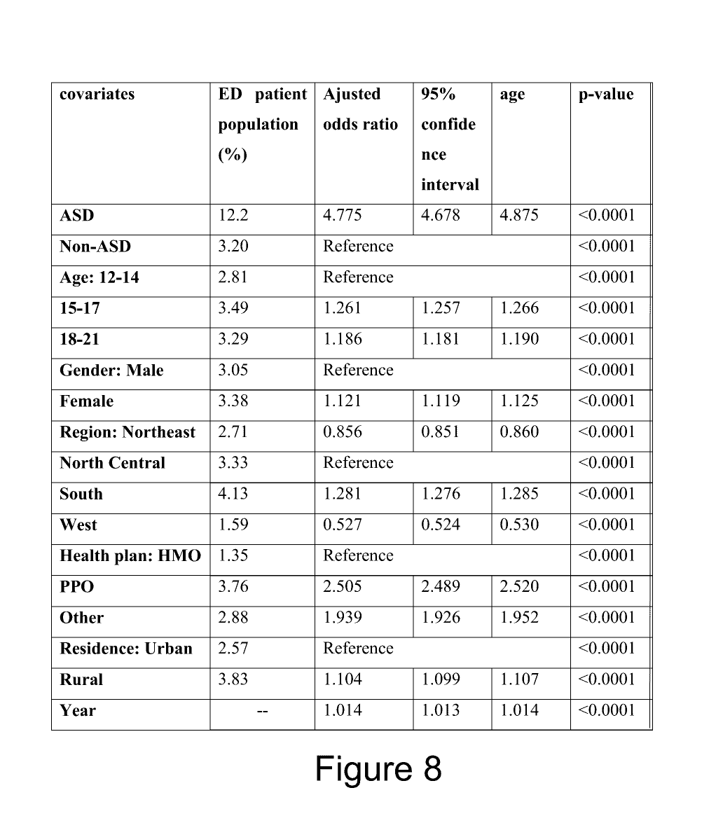

Multivariable logistic regression showed adolescents with ASD were associated with significant risk of have ED visit (Adjusted Odds Ratio, aOR=4.775; 95% Confidence Interval, CI=[4.678; 4.875]; p-value <0.0001) after adjusting for the other factors/confounders, such as age group, gender, US census region, geographic location, type of health plans and calendar year (FIG. 8). All the aforementioned covariates also had statistically significant associations with ED visit, though with more moderate effects.

Given the continuing difficulties with the transition from adolescence to adulthood for individuals with ASD, there is increasing interest to better understand the challenges of their teen years (Bureau of Autism Services 2011; Marcus et al., Handbook of autism and developmental disorders. 1997, 2^{nd} ed., pp. 631-649, New York: Wiley). The primary goal of this study was to examine emergency department (ED) utilization as a preliminary step in exploring the negative health experiences of adolescents with ASD. A large private insurance claims database (MarketScan®) was utilized to compare ED visits for adolescents with and without ASD for the years 2005 to 2013. It was hypothesized that adolescents with ASD would have significantly more ED visits than adolescents without ASD. Furthermore, adolescents with ASD who were older and living in rural communities were hypothesized to be at increased risk.

The results indicate support of the hypothesis. Over the study period, the percentage of adolescents with ASD who had an ED visit increased from 3.1% in 2005 to 15.8% in 2013 while the percentage of adolescents without ASD with ED visits remained around 3% for the same time period. Adolescents with ASD above the age of 14 accessed the ED at significantly higher rates than those aged 12- to 14-years-old; this discrepancy was not seen in the non-ASD cohort. Individuals with ASD residing in rural areas were slightly more likely to access the ED than individuals with ASD living in urban areas over the entire study period. There was no difference in ED utilization between individuals living in rural as compared to urban areas for the non-ASD cohort. Post hoc analysis revealed increased ED utilization for adolescent ASD females as well as non-ASD females. The proportion of adolescent ED patients who received behavioral health services during their visit increased from 2.6% to 5.9% for the non-ASD cohort over the study period; the ASD cohort demonstrated a comparable increase from 11.9% to 21.6%. Multivariable logistic regression demonstrated that adolescents with ASD accessed ED services over four times as often as adolescents without ASD after adjusting for confounders such as age, gender, setting, and calendar year.

These results coincide with previous research. Numerous studies have documented high costs of medical care for children and adolescents with ASD (Croen et al., Pediatrics, 2006, 118:e1203-e1211; Peacock et al. J Dev Behav Pediatr., 2010, 33:2-8; Shimabukuro et al., J Autism Dev Disord., 2008, 38:546-552). More salient to this line of inquiry is the previous finding that Deavenport-Saman and colleagues reported higher ED utilization for children and adolescents with ASD as compared to those without ASD, although they reported the increase as being more modest than this study's findings. This increased utilization encompasses both medical (Cohen-Silver et al., Clin Pediatr (Phila). 2014, 53:1134-1138; Deavenport-Saman et al., Matern Child Health J. 2016, 20:306-314; Vohra et al., J Autism Dev Disord. 2016, 46:1441-1454) and psychiatric (Iannuzzi et al., J Autism Dev Disord. 2015, 45:1096-1102; Kalb et al., Pediatr Emerg Care. 2012, 28:1269-1276; Wharff et al., Pediatr Emerg Care. 2011, 27:483-489) referrals. While adolescents in general are presenting with more psychiatric concerns (Sills and Bland, Pediatrics, 2002, 110:e40), this is particularly true of adolescents with ASD (Kalb et al., Pediatr Emerg Care. 2012, 28:1269-1276). The current study took this line of research further by examining a large, national database over nine years. Larger differences in ED utilization between adolescents with and without ASD were discovered. Over the study period, ED utilization for adolescents with ASD demonstrated significant increases while ED utilization for adolescents without ASD demonstrated only a modest increase.

### Implications for Research and Practice

The 2015 National Autism Indicators Report found that 60% of youth on the autism spectrum had at least two additional health or mental health conditions with 75% of youth taking at least one prescription medication to treat these conditions. This report also found a sharp drop off in needed services for youth with ASD between high school and their early 20s such that 26% of young adults on the autism spectrum receive no services and 37% of young adults with ASD are not employed or attending higher education. The National Longitudinal Transition Study—2 published data indicating that 50% of adolescents with ASD behaved in ways at home that resulted in parental disciplinary action and 59% of adolescents with ASD have difficulty controlling their temper when arguing with peers who are not their siblings (Wagner et al., After high school: A first look at the postschool experiences of youth with disabilities. A report from the National Longitudinal Transition Study-2 (NLTS2), 2005). Increasing needs during adolescence paired with declining services place youth with ASD to be vulnerable to physical and mental health crises.

Previous work has identified several predictors of ED utilization in children and adolescents with ASD, including day and time (Cohen-Silver et al., Clin Pediatr (Phila). 2014, 53:1134-1138) and type of insurance (Deavenport-Saman et al., Matern Child Health J. 2016, 20:306-314; Kalb et al., Pediatr Emerg Care. 2012, 28:1269-1276). Additionally, approximately one-fourth of children and adolescents with ASD have been found to make repeated visits to the ED, often within weeks of each other (Cohen-Silver et al., Clin Pediatr (Phila). 2014, 53:1134-1138). What is most concerning from the current findings is the consistent sharp increases in ED utilization by adolescents with ASD over the study period particularly as this was not seen in non-ASD youth. In 2005, ASD youth were only slightly more likely to utilize the ED compared to non-ASD youth. In 2013, ASD youth were five times more likely to visit the ED. This increased utilization could only be partially accounted for by increased identified ASD youth; the ASD cohort varied from a low of 5,740 (2007) to a high of 14,547 (2011). As this database is comprised of claims from insurance plans of large employers and health plans, some of the variation over the study period may be due to the financial crisis of 2007 and subsequent recovery. However, the proportion of ASD youth to the total adolescent population in the database remained relatively constant for this time period and the prevalence rate in this sample is below the CDC reported rates throughout the study period. However, greater numbers of ASD youth may be overwhelming healthcare resources not equipped to meet their needs.

Based on this analysis, not only are ASD youth utilizing the ED at disproportionate rates, they are accessing the ED for behavioral health concerns at increasing rates, outpacing non-ASD youth. By 2013, over 20% of ASD youth were accessing ED services for a readily identified behavioral health concern in the claims data. This appears to be a significant factor as the changes in behavioral health presenting concerns mirror the overall increased ED utilization. Adolescents with ASD who present for emergent psychiatric care appear to have difficulty accessing community based mental health services (Krauss et al., Ment Retard. 2003, 41:329-339; Soto et al., J Clin Psychiatry. 2009, 70:1164-1177). Better access to behavioral health services designed for adolescents with ASD, such as social skills training and specialized care coordination, appears to be a critical need. Greater understanding of service utilization before and after ED visits for youth with ASD is needed to craft more impactful interventions for individuals at risk.

This study found that the number of visits to the ED for adolescents with ASD significantly increased over time in comparison to age-matched typically developing peers. Overall, adolescents with ASD access the ED over four time more often than adolescents without ASD. Middle to late adolescence and residing in rural areas appeared to reflect higher rates of ED utilization in comparison to early adolescence and residing in urban areas. Additional analyses identified higher ED utilization in females with ASD as compared to males with ASD, as well as a significant increase in behavioral health visits in the ED over time. Adolescents with ASD do not appear to be adequately supported through the transition to adulthood as they experience more social and communication difficulties often while dealing with new onset mental health conditions. This may be particularly true for older youth, those residing in rural settings, and for adolescent females with ASD.

Next steps for research related to ED utilization in adolescents and young adults with ASD include examining databases which contain records for ED utilization in individuals with ASD who have public insurance as this was not included in the current data. The addition of a comparison group such as adolescents with Attention-Deficit/Hyperactivity Disorder (ADHD) in addition to typically developing adolescents may provide information about ED utilization in a separate, but related chronic, behavioral health concern. Additionally, further profiling the characteristics of ASD patients who had ED visits is an important research question. For example, medical records within 12 months before their ED visits can be examined to shed light on predictors to ED visits. Additional variables of question include rates of recidivism, subsequent inpatient hospitalization rates, and length of hospital stays for individuals with ASD as compared to a typically developing cohort. The type of episode of disease or injury with a high-incidence ratio in individuals with ASD as compared to typically developing peers is also of interest. Finally, given the findings in this study related to the increased presence of females with ASD in the ED, there is specific interest in identifying the characteristics of their ED episodes and factors in which they may differ from male adolescents and young adults counterparts.

### Example 3

### A Profile on HealthCare Utilization during the first year of life in Children with Autism Spectrum Disorder

An analysis of ED utilization by children who were later diagnosed with ASD shows that ASD children had distinct patterns of healthcare utilization even during their infancy: they used more healthcare services than non-ASD children, they seemed sicker and more vulnerable as early as within 6 months after birth, well before any clinically identifiable ASD symptoms were able to manifest, and some children with ASD appeared to have more lingering problems since birth.

### The Materials and Methods are now Described

### Data Source

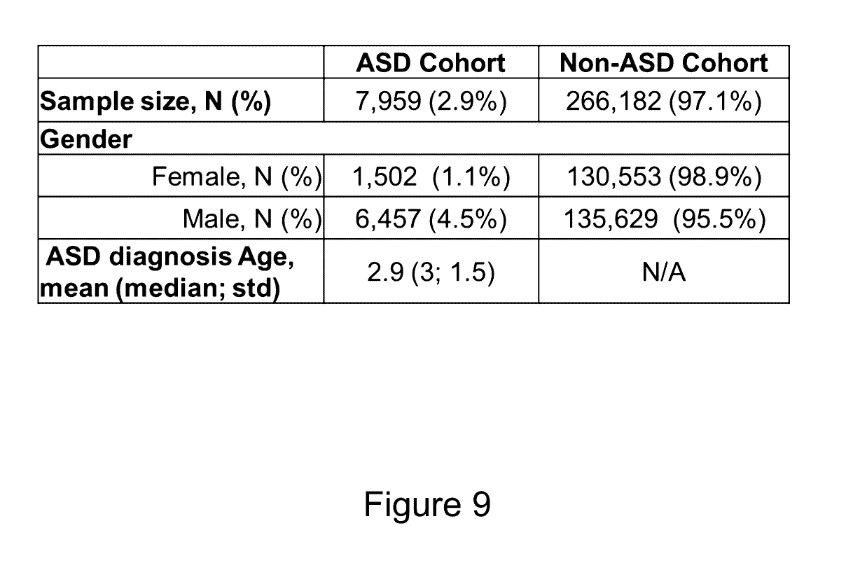

The MarketScan® Commercial Claims and Encounters database (Truven Health Analytics) was used as a source for a retrospective cohort study using data from 2005-2013. The individuals included in the study consisted of children who were born in this period. The individuals were followed for at least one year. The ASD cohort consisted of children with at least two separate diagnoses of ASD (ICD 9 codes 299.0x and 299.8x), and the control cohort consisted of children with no ASD diagnosis before the age of 6. The sample size, the breakdown of the cohorts by age, and the median age of ASD diagnosis is provided in FIG. 9.

### Statistical Analysis

Descriptive analyses were used for exploring cohort demographics and the distributions of variables. Univariate association between each factor of interest and the outcome variable was examined using Chi-squared test/Fisher's exact test for categorical variables, and t-test/Wilcoxon rank sum test for continuous variables. Multivariable analysis was performed by using general linear model or logistic regression model.

### The Experimental Results are now Described

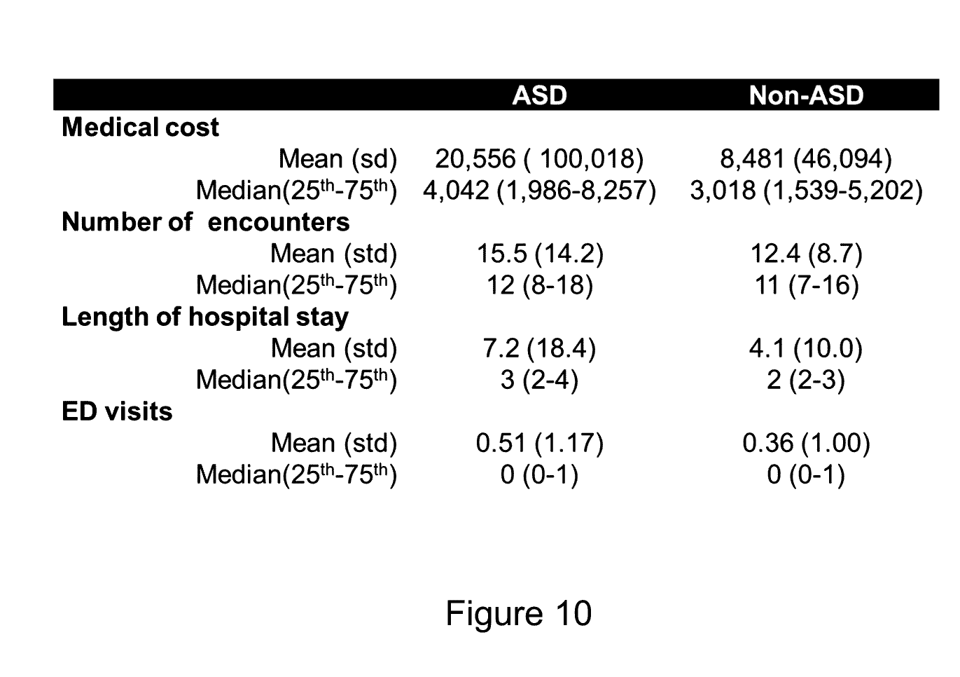

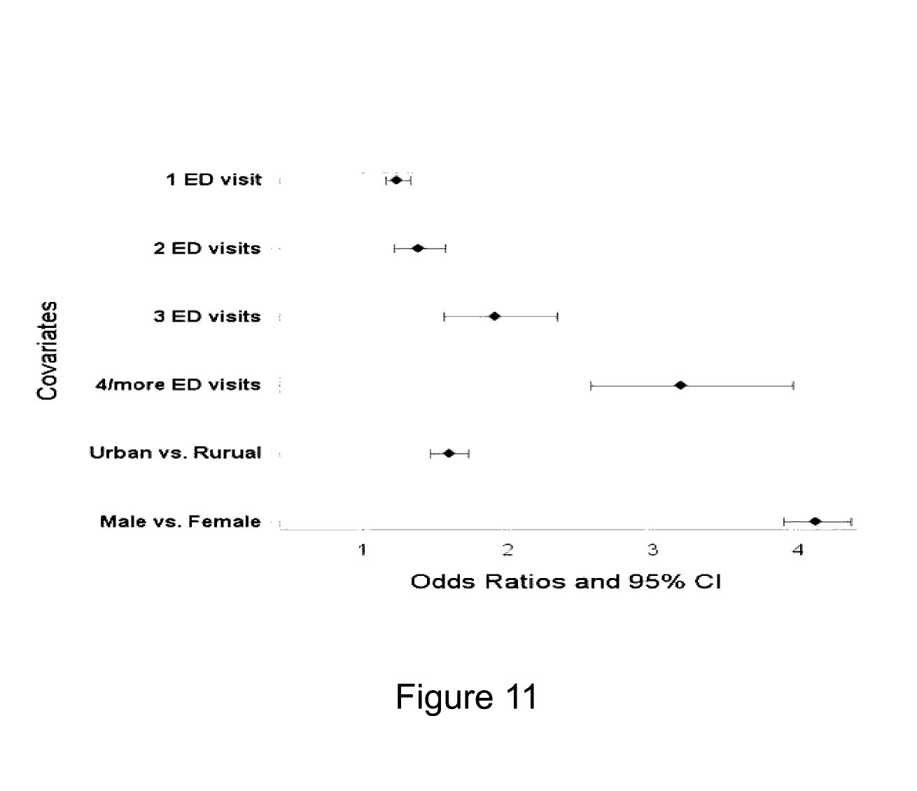

Healthcare utilization measures during the first year of life showed significant differences (p-value <0.0001) between children with ASD and those without (FIG. 10). On average, ASD children had higher healthcare costs, more in-/out-patient encounters, longer stay in hospitals, and more ED visits. The frequency of ED visits in the first 6 months is significantly associated with the risk of being diagnosed with ASD later on, after controlling for gender, geographic region and residence (urban vs. rural) (FIG. 11). Compared with children without any ED visit in the first 6 months, the adjusted odds ratios (95% confidence intervals) of ASD risk for 1, 2, 3, and >3 ED visits were 1.24 (1.16-1.32), 1.38 (1.22-1.57), 1.91 (1.56-2.34) and 3.19 (2.57-3.96), respectively.

The data indicate that infants with more ED visits (3, 4 or more during first 6 months) had a high likelihood of later being diagnosed with ASD.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for assessing the severity of ASD in a subject, the method comprising:
a. determining the incidence of at least one medical condition in a subject,
b. comparing the incidence of the at least one medical condition with a comparator control, and
c. assessing the subject's degree of ASD severity when the level of the at least one medical condition is present at a statistically significant amount when compared with the comparator control.

2. The method of claim 1, wherein the at least one medical condition is selected from the group consisting of unspecified hearing loss, Otitis Media, epilepsy, lack of coordination, influenza with other manifestation, patent ductus arteriosus, dysphagia, abdominal pain, convulsion, ear pain, teething syndrome, delayed milestone, weight/metabolism issue, disorder due to short gestation/low weight, lingering issue from labor complications, respiratory abnormalities, asthma, delay in development, preterm infants, rash and other non-specified skin eruption, constipation, diarrhea, contact dermatitis and other eczema, atopic dermatitis and related conditions, lack of normal physiological development, vomiting, acute laryngitis and trachetitis, feeding difficulty, colitis, viral infection, acute pharyngitis, cough, GERD, fever, acute upper respiratory infection, and any combination thereof.

3. The method of claim 1, wherein the subject is a child.

4. The method of claim 3, wherein the child is less than 6 months old.

5. The method of claim 3, wherein the child is less than 90 days old.

6. The method of claim 1, wherein the incidence of the at least one medical condition is characterized as a health profile.

7. The method of claim 6, wherein an assessment of ASD comprises a health profile including epilepsy and otitis media in a child less than 6 months old.

8. The method of claim 6, wherein the health profile is recorded over time as the subject ages.

9. The method of claim 6, wherein an assessment of ASD comprises a health profile recording at least two incidences of: respiratory system disease, abnormal weight or physical development, digestive system disorder, disorder relating to short gestation or low body weight, or neonatal labor complication.

10. The method of claim 1, wherein at least one step is carried out using a computer.

11. The method of claim 10, wherein the computer uses data from an Electronic Medical Records (EMR) database.

12. The method of claim 11, further comprising a step of generating a notice when an assessment of significant ASD severity is made.

13. The method of claim 10, wherein at least one step is carried out using a software application or a website.

14. The method of claim 13, wherein the software application is subscription-based.

15. The method of claims 13, wherein the method of assessment is carried out once per transaction.

16. The method of claim 1, further comprising the step of providing the subject with a medical treatment based upon ASD severity.

17. The method of claim 16, wherein the treatment step is early intervention therapy to improve communication, locomotion, and socialization ability in a subject that is less than 36 months of age.

18. The method of claim 16, wherein the treatment step is selected from the group consisting of early intervention therapy, occupational therapy, behavioral therapy, communication therapy, dietary therapy, drug therapy, complementary medicine therapy, alternative medicine therapy, and any combination thereof.

19. The method of claim 1, further comprising the step of providing ASD educational information based upon ASD severity.

20. The method of claim 1, wherein the assessment of ASD severity is updated by performing the method periodically.

21. The method of claim 1, wherein the comparator control is periodically updated.

22. The method of claim 1 further comprising the step of diagnosing said subject with ASD based on said subject's level of incidence of said one or more medical conditions compared to said comparator control.

23. A method of diagnosing ASD in a subject, the method comprising:
a. determining the incidence of at least one medical condition in a subject,
b. comparing the incidence of the at least one medical condition with a comparator control, and
c. diagnosing the subject with ASD when the level of the at least one medical condition is present at a statistically significant amount when compared with the comparator control.

24. The method of claim 23, wherein the at least one medical condition is selected from the group consisting of unspecified hearing loss, Otitis Media, epilepsy, lack of coordination, influenza with other manifestation, patent ductus arteriosus, dysphagia, abdominal pain, convulsion, ear pain, teething syndrome, delayed milestone, weight/metabolism issue, disorder due to short gestation/low weight, lingering issue from labor complications, respiratory abnormalities, asthma, delay in development, preterm infants, rash and other non-specified skin eruption, constipation, diarrhea, contact dermatitis and other eczema, atopic dermatitis and related conditions, lack of normal physiological development, vomiting, acute laryngitis and trachetitis, feeding difficulty, colitis, viral infection, acute pharyngitis, cough, GERD, fever, acute upper respiratory infection, and any combination thereof.

25. The method of claim 23, wherein the subject is a child.

26. The method of claim 25, wherein the child is less than 6 months old.

27. A method for assessing the risk of developing Autism spectrum disorder (ASD) in a subject, the method comprising:
a. determining the incidence of at least one medical condition in a subject,
b. comparing the incidence of the at least one medical condition with a comparator control, and
c. assessing the subject's risk of developing ASD when the level of the at least one medical condition is present at a statistically significant amount when compared with the comparator control.

28. The method of claim 27, wherein the at least one medical condition is selected from the group consisting of unspecified hearing loss, Otitis Media, epilepsy, lack of coordination, influenza with other manifestation, patent ductus arteriosus, dysphagia, abdominal pain, convulsion, ear pain, teething syndrome, delayed milestone, weight/metabolism issue, disorder due to short gestation/low weight, lingering issue from labor complications, respiratory abnormalities, asthma, delay in development, preterm infants, rash and other non-specified skin eruption, constipation, diarrhea, contact dermatitis and other eczema, atopic dermatitis and related conditions, lack of normal physiological development, vomiting, acute laryngitis and trachetitis, feeding difficulty, colitis, viral infection, acute pharyngitis, cough, GERD, fever, acute upper respiratory infection, and any combination thereof.

29. The method of claim 27, wherein the subject is a child.

30. The method of claim 29, wherein the child is less than 6 months old.

31. A system for diagnosing ASD in a subject, the system comprising a component to assess the presence of at least one medical condition in a subject.

32. The system of claim 31, wherein the at least one medical condition is selected from the group consisting of unspecified hearing loss, Otitis Media, epilepsy, lack of coordination, influenza with other manifestation, patent ductus arteriosus, dysphagia, abdominal pain, convulsion, ear pain, teething syndrome, delayed milestone, weight/metabolism issue, disorder due to short gestation/low weight, lingering issue from labor complications, respiratory abnormalities, asthma, delay in development, preterm infants, rash and other non-specified skin eruption, constipation, diarrhea, contact dermatitis and other eczema, atopic dermatitis and related conditions, lack of normal physiological development, vomiting, acute laryngitis and trachetitis, feeding difficulty, colitis, viral infection, acute pharyngitis, cough, GERD, fever, acute upper respiratory infection, and any combination thereof.

33. The system of claim 31, wherein the subject is a child.

34. The system of claim 33, wherein the child is less than 6 months old.

35. A method for diagnosing risk of developing ASD in a subject, the method comprising:
a. determining the number of emergency department (ED) visitations by a subject during the first six months of life, and
b. diagnosing the subject as having an increased risk of developing ASD when the number of emergency department visitations by the subject is greater than two.

36. The method of claim 35 further comprising the step of providing the subject with an ASD evaluation or ASD therapy based upon increased ED utilization.

37. The method of claim 36, wherein the therapy is selected from the group consisting of early intervention therapy, pivotal response treatment (PRT), verbal behavior (VB) therapy, applied behavioral analysis (ABA), discrete trial teaching (DTT), floortime (DIR), relationship development intervention (RDI), training and education of autistic and related communication handicapped children (TEACCH), social communication/emotional regulation/transactional support (SCERTS), speech-language therapy (SLT), occupational therapy (OT), sensory integration (SI), physical therapy (PT), social skills therapy, picture exchange communication system (PECS), auditory integration therapy (AIT), gluten free casein free diet (GFCF), early start Denver model (ESDM), behavioral therapy, communication therapy, dietary therapy, drug therapy, complementary medicine therapy, alternative medicine therapy or any combination thereof.

38. A method for diagnosing risk of developing ASD in a subject, the method comprising:
a. determining the number of ED visitations by a subject,
b. comparing the number of ED visitations with a comparator control, and
c. diagnosing the subject as having an increased risk of ASD when the number of ED visitations by the subject is statistically significantly greater when compared with the comparator control.

39. The method of claim 35 further comprising the step of providing the subject with a medical therapy based upon ED utilization.

40. The method of claim 39, wherein the therapy is selected from the group consisting of occupational therapy, behavioral therapy, communication therapy, dietary therapy, drug therapy, complementary medicine therapy, alternative medicine therapy, and any combination thereof
